(19) United States
(12) Patent Application Publication
(10) Pub. No.: US 2009/0181362 A1
(43) Pub. Date: Jul. 16, 2009

(54) METHOD FOR DETERMINATION OF RECOGNITION SPECIFICITY OF VIRUS FOR RECEPTOR SUGAR CHAIN

(76) Inventors: Yasuo Suzuki, Kyoto (JP); Akira Asai, Shizuoka (JP); Takashi Suzuki, Shizuoka (JP); Kazuya Hidari, Shizuoka (JP); Takeomi Murata, Shizuoka (JP); Taiichi Usui, Shizuoka (JP); Sou Takeda, Chiba (JP); Kohei Yamada, Chiba (JP); Toshitada Noguchi, Chiba (JP)

Correspondence Address: WINSTON & STRAWN LLP;PATENT DEPARTMENT, 1700 K STREET, N.W., WASHINGTON, DC, 20006, US

(21) Appl. No.: 12/065,469

(22) PCT Filed: Aug. 29, 2006
(86) PCT No.: PCT/JP2006/316928

§ 371 Date: Mar. 11, 2009

(30) Foreign Application Priority Data
Sep. 2, 2005 (JP) 2005-255730
Feb. 27, 2006 (JP) 2006-050943

(51) Int. Cl. C12Q 1/70; C07H 7/02

(52) U.S. Cl. 435/5; 536/17.9

(57) ABSTRACT

A method is provided in which the recognition specificity of a virus for a receptor sugar chain can be easily determined with a simple instrument or apparatus. This method for determining the recognition specificity of a virus for a receptor sugar chain or for determining the change in a host infected in accordance with the mutation of virus includes the steps of bringing a sample of the virus into contact with a support having a polymer with sialo-oligosaccharide immobilized on the surface thereof; and assaying the degree of binding therein to determine the recognition specificity of the virus for the receptor sugar chain and to determine the change in a host range. The method is suitable for the surveillance of a virus.

### TECHNICAL FIELD

The present invention is related to a method for determining the recognition specificity of a virus for a receptor sugar chain, a new polymer with sialo-oligosaccharide and a support which can be used in the method, and an effective manufacturing method thereof.

### BACKGROUND ART

There are numerous symptoms of the influenza, from light symptoms like a common cold to severe (life threatening) symptoms like the Spanish flu. In addition, the influenza is a zoonotic disease, therefore, the avian influenza is recently becoming a major problem. It is known that the range of hosts of influenza viruses extends to many animals species. For example, wild waterfowl such as ducks, domestic fowl such as turkeys, chickens, and quails, pigs, horses, cows, ferrets, whales and seals as well as humans can all become hosts for viruses A.

The coat of the influenza virus is covered with projections of two types of enzyme proteins one of which is HA (hemagglutinins) and the other of which is NA (neuraminidase). HA is a hemagglutininating antigen and at the time of attachment, and invasion to a host cell, it binds with a receptor sugar chain containing sialic acid on the surface of the cell and plays an important role when a viral particle is ingested within the cell.

The antigenecity of an influenza virus is decided by a combination of HA and NA and is divided broadly into three types A, B and C. There are four further subtypes such as the Hong Kong strain which are known among the A type. Conventionally, it is known that a different subtype appears in cycles of about ten years and even within the same subtype, the antigenecity changes little by little every year (antigen shift) in the A type. As a result, it is difficult to produce a vaccine which is completely suitable for an antigenic form and its prevention effects have become problematic.

Meanwhile, among classification of the types of influenza virus, other than the above stated category according to antigenicity, there is also a category according to the differences of binding ability of the influenza virus to the receptor sugar chain (non patent document 1). This category is based on the differences of the mode of binding to sialic acid at an end of a receptor sugar chain and also on the differences in the degrees of recognition, binding ability or affinity of the influenza virus to a receptor sugar chain.

For example, the highly pathogenic avain influenza viruses (such as the H5N1 subtype and H9N1, H7N7) strongly recognize the binding mode of [SAα2-3Galβ- (SA: sialic acid)], but the recognition, biding ability or affinity is low for the binding mode of [SAα2-6Galβ-]. On the other hand, the human influenza A virus and human influenza B virus strongly recognize the binding mode of [SAα2-6Galβ-] but the recognition, biding ability or affinity is low for the binding mode of [SAα2-3Galβ-].

The most effective method for judging the ability of an avian influenza virus to infect humans is the method of recognizing the binding ability of the influenza virus to the receptor sugar chain. That is, even in the case where the avian influenza virus has infected a human that does not mean that a change in the host range will be reflected in a mutation of a gene. However, because a variation in the binding ability to a receptor sugar chain is essential for infection, if the recognition specificity of an influenza virus for a receptor sugar chain or its variation can be easily determined, not only can the type of the influenza virus be determined but also a change of a host infected due to a mutation of the virus or the possibility of a large spread can be predicted.

Conventionally, the Resonant Mirror Detection method is used as a method for determining the recognition specificity of a virus for a receptor sugar chain (patent document 1). In this method, a receptor sugar strain for an influenza virus is immobilized within a cuvette of the Resonant Mirror apparatus and an influenza virus sample is made to react with the receptor sugar chain. Then, a change in the resonant angle which occurs by the binding of the receptor sugar chain and the influenza virus is expressed in a binding curve and the response strength is monitored. It is assumed that the recognition specificity of a virus for a receptor sugar chain can be determined by the strength of this response.

Nevertheless, it is difficult to immobilize a receptor sugar chain to a support in this method. That is, a glycoceramide (sialyl (2-3) neolactotetraosylceramide (avian type), sialyl (2-3) lactotetraosylceramide (avian type), sialyl (2-6) neolactotetraosylceramide (human type) and sialyl (2-6) lactotetraosyiceramide (human type) etc.) is used as a receptor sugar chain, a glycolipid which does not bind with the influenza virus is further mixed with the glycoceramide and an immobilized receptor sugar chain is prepared by an extremely cumbersome and complicated method in which this glycolipid mixture is immobilized to the bottom surface within the cuvette. Furthermore, it is necessary to use special and large apparatus of the Resonant Mirror apparatus. As a result, although it can be used in large scale research facilities, it is difficult to use in places where patients arise such as airports, poultry farms and stations etc. or in clinical places such as hospitals.

Recently, it is pointed that the avian influenza virus which is highly toxic will be spreading worldwide and the possibility of a pandemic may be occur by mutation of the virus into a virus (new influenza virus) which infects from a human to another human. As a result, the rapid development of a method which can easily determine the recognition specificity of an influenza virus for a receptor sugar chain using inexpensive and simple instruments is being eagerly desired.

Non patent document 1: Sugar chain recognition process in virus infections (Yasuo Suzuki, Biochemistry Volume 76, No. 3, pp. 227-233, 2004))

### Patent Document 1: Japanese Laid Open Patent Publication

### Patent Document 2: Japanese Laid Open Patent Publication 2003-73397

### Patent Document 3: Japanese Laid Open Patent Publication H10-310610

### Patent Document 4: Japanese Laid Open Patent Publication 2003-535965

### Patent Document 5: Japanese Laid Open Patent Publication H11-503525

### Patent Document 6: Japanese Laid Open Patent Publication 2004-115616

### DISCLOSURE OF THE INVENTION

### Problems to be Solved

The inventors of the present invention tried to develop a method for easily determining the recognition specificity of an influenza virus for a receptor sugar chain using inexpensive and simple instruments and attempted an application of an immunologic assay such as the ELISA method and immunochromatography method.

However, in order to establish a method for determining the recognition specificity of an influenza virus for a receptor sugar chain by applying an immunologic assay, it is realized that there is a need to solve the following types of problems. (1) selection of a compound containing a receptor sugar chain (problem 1), (2) establishment of an efficient manufacturing method of a compound containing a receptor sugar chain (problem 2), (3) establishment of a method for immobilizing a compound containing a receptor sugar chain to a support (problem 3), (4) determining the recognition specificity of an influenza virus for a receptor sugar chain from an assay result, predicting a change in host range and confirming its usability as a reagent or kit for surveillance use (problem 4).

More specifically, there are the following problems associated with each of the above problems and without solving these problems it is impossible to determine the recognition specificity of an influenza virus for a receptor sugar chain.

### [Problem 1]

Conventionally, although a variety of compounds containing a receptor sugar chain with which an influenza virus can be bound have been reported (Patent Documents 2-4), there have been no reports of compounds containing a receptor sugar chain which are suitable in a method for determining the recognition specificity of an influenza virus for a receptor sugar chain. Furthermore, it is essential that an inactivated virus sample can be used when consideration is given to safety during an assay. However, even in the case where an inactivated virus sample is used preferably without being concentrated, it is still unclear what kind of compound containing a receptor sugar chain can bind with such a sample.

### [Problem 2]

The method disclosed in Patent Document 2 is given as a method for synthesizing a polyglutamic acid with sialo-oligosaccharide as one example of a compound containing a receptor sugar chain. According to this method, p-nitro phenyl N-acetyl-β-lactosaminide is synthesized by utilizing the glycosyltransferase reaction of β galactosidase and the p-nitro phenyl group is reduced to a p-amino phenyl group. Then, it is coupled with polyglutamic acid and by sialylating oligosaccharide units using a sialytransferase from rats, the desired polymer with sialo-oligosaccharide was obtained.

However, this method was not an industrially satisfactory method and has the following disadvantages. (1) The synthetic yield is extremely poor, (2) enzymes from microorganisms cannot be used as glycosyltransferase due to the specificity of substrate and only expensive enzymes from animals, the preparation of which is extremely troublesome, can be used, (3) because it is difficult to control the introduction rate of sialo-oligosaccharides to polyglutamic acid residues, there is a need for a large surplus of p-amino phenylated oligosaccharide or in the case where a sialo-oligosaccharide is directly coupled with a polyglutamic acid it is necessary to protect a carboxyl group in order to reduce side reactions, (4) because a polyglutamic acid structure is degraded by a protease or peptidase, there is a need to use a purified enzyme as the sialytransferase to be used.

### [Problem 3]

A method in which an appropriate linker is used as a method for immobilizing a compound containing a receptor sugar chain to a support is generally used (Patent Documents 5 and 6). However, a method which uses a linker is not simple and because chemical and undesired side reactions occur it is not a desirable method. Furthermore, in the case where a polyglutamic acid with sialo-oligosaccharide is used as a polymer with receptor sugar chain is given as an example, a binding method of the polyglutamic acid with sialo-oligosaccharide to a support has not been reported.

### [Problem 4]

Until now, a reagent or a kit which can determine the recognition specificity of a virus for a receptor sugar chain or predict a change in a host infected by a virus mutation has not been reported or commercially available.

### Means for Solving the Problems

The inventors of the present invention gained the following knowledge as a result of repeated keen examinations in order to solve the above stated problems and completed the present invention. (1)

For catching a virus by applying an immunologic assay for example, a polymer with sialo-oligosaccharide which is a composite of a sialo-oligosaccharide and a polymer or more particularly a polyglutamic acid with sialo-oligosaccharide is more suitable than a sialo-oligosaccharide by itself and can also be used for an inactivated virus sample, (2) this polyglutamic acid with sialo-oligosaccharide can be efficiently synthesized by changing a synthesis scheme into a scheme in which after synthesizing a trisaccharide it is coupled with a polyglutamic acid at the final stage, (3) as a method of immobilizing the polyglutamic acid with sialo-oligosaccharide to a support, not by binding with an appropriate linker but by bringing a solution which includes a polymer with sialo-oligosaccharide into contact with a support and irradiating it with ultra violet rays it is possible to efficiently immobilize the polyglutamic acid with sialo-oligosaccharide to the surface of the support. In addition (4) as a result of examining the binding specificity of a virus for a receptor sugar chain by applying the ELISA method using the immobilized polyglutamic acid with sialo-oligosaccharide, it is realized that the specificity of a virus for a receptor sugar chain can be determined and a change in a host infected by a virus mutation can be determined by measuring the degree of this binding. That is, as a result of the examination stated above, the inventors realized that by using a support wherein two or more different polymers with sialo-oligosaccharide are immobilized on the surface of the support or two or more supports each of which having a different polymer with sialo-oligosaccharide immobilized on each surface of the supports, bringing the sample of the virus into contact with each of the polymers with sialo-oligosaccharide, assaying the degree of binding therein and comparing the results, a change in the host infected caused by the virus mutation could be determined and completed the present invention. Therefore, the present invention is as follows below.

(1) A method for determining the recognition specificity of a virus for a receptor sugar chain including bringing a sample of the virus into contact with a support having a polymer with sialo-oligosaccharide immobilized on the surface thereof and assaying the degree of binding therein to determine the recognition specificity of the virus for the receptor sugar chain.
(2) A method for determining a change in a host range caused by a virus mutation including using a support wherein two or more different polymers with sialo-oligosaccharide are immobilized on the surface of the support or two or more supports each of which having a different polymer with sialo-oligosaccharide immobilized on each surface of the supports, bringing the sample of the virus into contact with each of the polymers with sialo-oligosaccharide, assaying the degree of biding therein and determining a change in the host range caused by the virus mutation by comparing the results.
(3) The determining method according to (1) or (2) stated above, wherein the sialo-oligosaccharide in the polymer with sialo-oligosaccharide is at least one sugar chain selected from a group consisting of sialyllacto-series type I sugar chain (SAα2-6(3)Galβ1-3GlcNAcβ1-), sialyllacto-series type II sugar chain (SAα2-6(3)Galβ1-4GlcNAcβ1-), sialylganglio-series sugar chain (SAα2-6(3)Galβ1-3GalNAcβ1-), and sialyl lactose sugar chain (SAα2-6(3)Gal1-4Glc-).
(4) The determining method according to (1) or (2) stated above, wherein the polymer in the polymer with sialo-oligosaccharide is a polyglutamic acid.
(5) The determining method according to (1) or (2) stated above, wherein assaying the degree of binding is an immunologic assay which uses an antivirus antibody against the virus.
(6) The determining method according to (1) or (2) stated above, wherein the virus sample is an influenza virus sample.
(7) A polymer with sialo-oligosaccharide having a γ-polyglutamic acid with which a sialo-oligosaccharide is coupled, and expressed in the following formula (I).

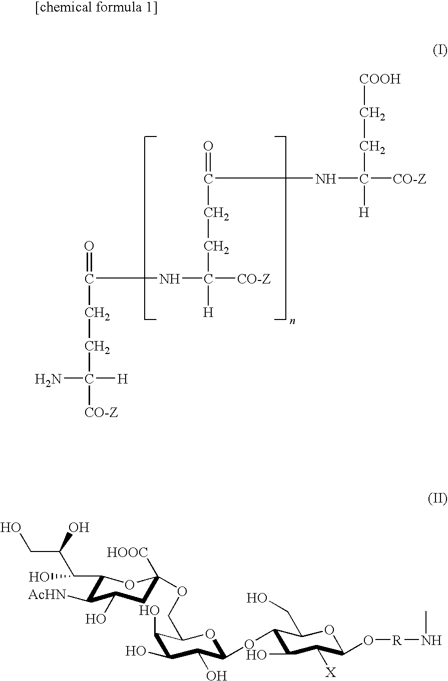

(In the formula (I), Z is a hydroxyl group or a sialo-oligosaccharide binding site expressed in the formula (II), n indicates an integer of 10 or more. In the formula (II), Ac is an acetyl group, X is a hydroxyl group or an acetyl amino group and R indicates a hydrocarbon).
(8) A polymer with sialo-oligosaccharide having a γ-polyglutamic acid with which a sialo-oligosaccharide is coupled, and expressed in the following formula (III).

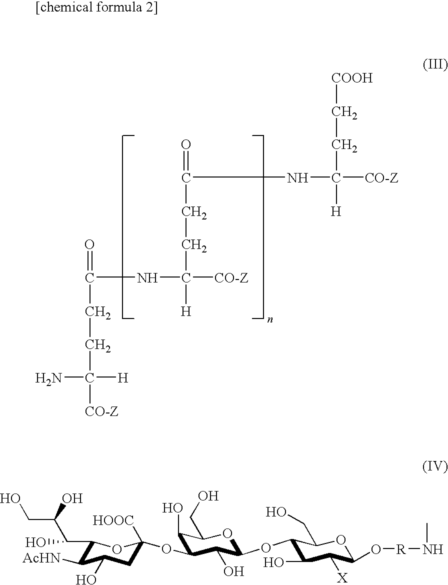

(In the formula (III), Z is a hydroxyl group or a sialo-oligosaccharide binding site expressed in the formula (IV), n indicates an integer of 10 or more. In the formula (IV), Ac is an acetyl group, X is a hydroxyl group or an acetyl amino group and R indicates a hydrocarbon).
(9) A polymer with sialo-oligosaccharide having an α-polyglutamic acid with which a sialo-oligosaccharide is coupled, and expressed in the following formula (V).

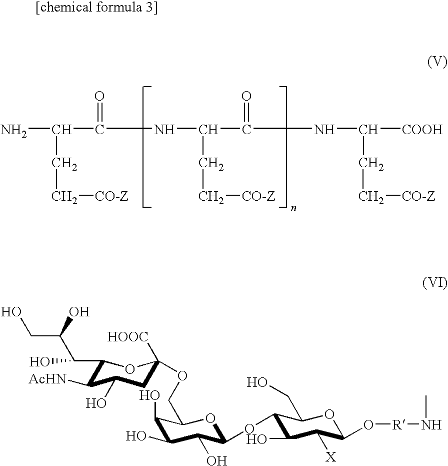

(In the formula (V), Z is a hydroxyl group or a sialo-oligosaccharide binding site expressed in the formula (VI), n indicates an integer of 10 or more. In the formula (VI), Ac is an acetyl group, X is a hydroxyl group or an acetyl amino group and R′ indicates a hydrocarbon except for phenylene).
(10) A polymer with sialo-oligosaccharide having an α-polyglutamic acid with which a sialo-oligosaccharide is coupled, and expressed in the following formula (VII).

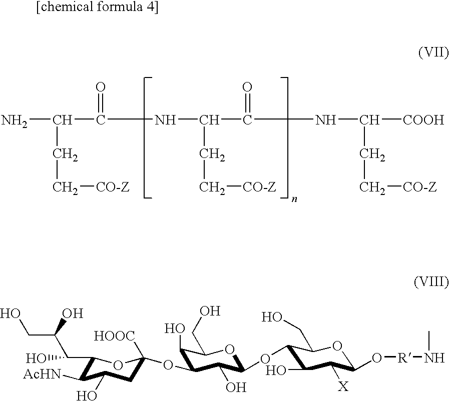

(In the formula (VII), Z is a hydroxyl group or a sialo-oligosaccharide binding site expressed in the formula (VIII), n indicates an integer of 10 or more. In the formula (VIII), Ac is an acetyl group, X is a hydroxyl group or an acetyl amino group and R′ indicates a hydrocarbon except for phenylene).
(11) A manufacturing method of a polymer with sialo-oligosaccharide including a process (1) wherein a desired sialo-oligosaccharide is synthesized using a glycosyltransferase, a process (2) wherein the sialo-oligosaccharide synthesized in process (1) is chemically coupled with a polyglutamic acid, a process (3) wherein a desired polymer with sialo-oligosaccharide is obtained by isolating and purifying the polymer with sialo-oligosaccharide synthesized in process (2).
(12) The manufacturing method according to (11) stated above, wherein the sialo-oligosaccharide is at least one sugar chain selected from a group consisting of sialyllacto-series type I sugar chain (SAα2-6(3)Galβ1-3GlcNAcβ1-), sialyllacto-series type II sugar chain (SAα2-6(3)Galβ1-4GlcNAcβ1-), sialylganglio-series sugar chain (SAα2-6(3)Galβ1-3GalNAcβ1-), and sialyl lactose sugar chain (SAα2-6(3)Gal1-4Glc-).
(13) A support used in the determining method of (1) or (2) stated above including a polymer with sialo-oligosaccharide immobilized on the surface of the support.
(14) A support comprising a polymer with sialo-oligosaccharide immobilized on the surface thereof by ultraviolet ray irradiation, in said polymer with sialo-oligosaccharide, at least one sialo-oligosaccharide selected from a group consisting of sialyllacto-series type I sugar chain (SAα2-6(3)Galβ1-3GlcNAcβ1-), sialyllacto-series type II sugar chain (SAα2-6(3)Galβ1-4GlcNAcβ1-), sialylganglio-series sugar chain (SAα2-6(3)Galβ1-3GalNAcβ1-), and sialyl lactose sugar chain (SAα2-6(3)Gal1-4Glc-) is coupled with a polyglutamic acid.
(15) The support according to (13) or (14) stated above, wherein the support contains a plurality of wells, and a plurality of polymers with sialo-oligosaccharide of different types being immobilized on the support.
(16) A kit used in the determining method of (1) or (2) stated above for determining the recognition specificity for a receptor sugar chain or a mutation of a virus having a support according to (14) stated above.
(17) The kit according to (16) stated above, wherein the support contains a plurality of wells, and a plurality of polymers with sialo-oligosaccharide of different types being immobilized on one support.
(18) The kit according to (16) stated above, wherein the kit contains two or more supports, and a polymer with sialo-oligosaccharide of different type being immobilized on each of the supports.
(19) The determining method according to (1) or (2) stated above, wherein the polymer in the polymer with sialo-oligosaccharide is an a polyglutamic acid.
(20) The determining method according to (6) stated above, wherein the influenza virus is an inactivated influenza virus.
(21) The polymer with sialo-oligosaccharide any one of (7) to (10) stated above, wherein a degree of polymerization in glutamic acid units is between 10 and 10,000.
(22) The polymer with sialo-oligosaccharide in any one of (7) to (10) stated above, wherein the introduction rate of sialo-oligosaccharides to glutamic acid residues is between 10% and 80%.
(23) The manufacturing method according to (11) stated above, wherein the polyglutamic acid is an α-polyglutamic acid or a γ-polyglutamic acid.
(24) The manufacturing method according to (11) stated above, wherein the degree of polymerization in glutamic acid units is between 10 and 10,000.
(25) The polymer with sialo-oligosaccharide according to (11) stated above, wherein the introduction rate of sialo-oligosaccharides to glutamic acid residues is between 10% and 80%
(26) The support according to (13) stated above, wherein the sialo-oligosaccharide in the polymer with sialo-oligosaccharide is at least one sugar chain selected from a group consisting of sialyllacto-series type I sugar chain (SAα2-6(3)Galβ1-3GlcNAcβ1-), sialyllacto-series type II sugar chain (SAα2-6(3)Gal1-4GlcNAcβ1-), sialylganglio-series sugar chain (SAα2-6(3)Gal1-3GalNAcβ1-), and sialyl lactose sugar chain (SAα2-6(3)Gal1-4Glc-).
(27) The support according to (13) or (14) stated above, wherein the polyglutamic acid is an α-polyglutamic acid or a γ-polyglutamic acid
(28) The support according to (13) or (14) stated above, wherein the support contains a plurality of wells.
(29) The kit according to (16) stated above, wherein the kit further includes an antiviral antibody against the virus.
(30) The manufacturing method of the support according to any one of (13) to (29) stated above, wherein a solution which includes a polymer with sialo-oligosaccharide is brought into contact with the support and in this state the support is irradiated with ultra violet rays, then the solution is removed so that the polymer with sialo-oligosaccharide is immobilized on the surface of the support.

### EFFECTS OF THE INVENTION

In this way, the determining method of the present invention is a method wherein a support to which a polymer with sialo-oligosaccharide, in particular a polyglutamic acid with sialo-oligosaccharide is immobilized, is used, and by bringing a virus into contact with this and by assaying the degree of binding therein by an immunologic method the recognition specificity of a tested virus for a receptor sugar chain is determined. The determining method of the present invention can be easily performed using simple instruments and according to the present invention, for example, in addition to being able to determine whether an influenza virus is a human infection type or an avian infection type it has become possible for the first time to predict a change in hosts infected due to a virus mutation or the possibility of spread.

Conventionally, various polymers with sialo-oligosaccharide itself or binding methods of sialo-oligosaccharides to supports have been reported (Patent Documents 2 to 6). However, there are no reports pronouncing that it is possible to determine the recognition specificity of a virus for a receptor sugar chain even when an inactivated virus sample is used, and it is not thought to be possible to determine. This has been achieved for the first time by the inventors of the present invention.

In addition, a polyglutamic acid with sialo-oligosaccharide and its manufacturing method of the present invention uses cheap materials and is an efficient method. As a result, it is possible to greatly reduce the cost of a polyglutamic acid with sialo-oligosaccharide, a support reagent to which it is immobilized and a kit of the present invention, it is possible to perform an examination without large expenditure and it is possible to use the kit, for example, of the present invention even in developing countries.

Furthermore, because it is possible to apply an immunologic assay method such as ELISA or a biological assay method for example in a support and kit in order to determine the recognition specificity of a virus for a receptor sugar chain of the present invention, it is easy to manufacture the support and the assay operation is also easy. As a result, the present invention can be performed anywhere, there is also no need to use large apparatus and it is possible to be used in a test facility to which samples have been brought from fields such as chicken farms, abbatoirs, hospitals, airports or stations and which is located near the fields.

### BRIEF DESCRIPTION OF THE DRAWINGS

FIG. 1 is a graph which shows a recognition specificity of an avian influenza A virus for a receptor sugar chain in one example of the present invention.

FIG. 2 is a graph which shows a recognition specificity of a human influenza A virus for a receptor sugar chain in the same example.

FIG. 3 is a graph which shows a recognition specificity of a human influenza B virus for a receptor sugar chain in the same example.

FIG. 4 is a graph which shows a recognition specificity of a human influenza A virus for a receptor sugar chain. ◯ shows the result of Poly (Neu5Acα2-6Lacβ5-animopentyl/γ-PGA), shows the result of Poly (Neu5Acα2-3Lacβ-5-animopentyl/γ-PGA) and Δ shows the result of Poly (Lacβ-5-animopentyl/γ-PGA).

FIG. 5 is a graph which shows a recognition specificity of an avian influenza A virus for a receptor sugar chain. ◯ shows the result of Poly (Neu5Acα2-6Lacβ-5-animopentyl/γ-PGA), shows the result of Poly (Neu5Acα2-3Lacβ-5-animopentyl/γ-PGA) and Δ shows the result of Poly (Lacβ-5-animopentyl/γ-PGA).

FIG. 6 is a graph which shows a recognition specificity of a human influenza A virus for a receptor sugar chain. ◯ shows the result of Poly (Neu5Acα2-6Lacβ-5-animopentyl/γ-PGA), shows the result of Poly (Neu5Acα2-3Lacβ-5-animopentyl/γ-PGA) and Δ shows the result of Poly (Lacβ-5-animopentyl/γ-PGA).

FIG. 7 is a graph which shows a recognition specificity of an avian influenza A virus for a receptor sugar chain. ◯ shows the result of Poly (Neu5Acα2-6Lacβ-5-animopentyl/γPGA), shows the result of Poly (Neu5Acα2-3Lacβ-5-animopentyl/γ-PGA) and Δ shows the result of Poly (Lacβ-5-animopentyl/γ-PGA).

FIG. 8 is a graph which shows a recognition specificity of a human influenza A virus for a receptor sugar chain. ◯ shows the result of more high-molecular-weight Poly (Neu5Acα2-6Lacβ-5-animopentyl/γ-PGA), and shows the result of more high-molecular-weight Poly (Neu5Acα2-3Lacβ-5-animopentyl/γ-PGA).

FIG. 9 is a graph which shows a recognition specificity of an avian influenza A virus for a receptor sugar chain. ◯ shows the result of more high-molecular-weight Poly (Neu5Acα2-6Lacβ-5-animopentyl/γ-PGA), and shows the result of more high-molecular-weight Poly (Neu5Acα2-3Lacβ-5-animopentyl/γ-PGA).

FIG. 10 is a graph which shows a recognition specificity of a human influenza A virus for a receptor sugar chain. ◯ shows the result of Poly (Neu5Acα2-6LacNAcβ-p-animophenyl/γ-PGA), shows the result of Poly (Neu5Acα2-3LacNAcβ-p-animophenyl/γ-PGA) and □ shows the result of Poly (Neu5Acα2-6LacNAcβ-p-animophenyl/α-PGA) and ▪ shows the result of Poly (Neu5Acα2-3LacNAcβ-p-animophenyl/α-PGA).

FIG. 11 is a graph which shows a recognition specificity of the avian influenza A virus for a receptor sugar chain in the above stated example. ◯ shows the result of Poly (Neu5Acα2-6LacNAcβ-p-animophenyl/γ-PGA), shows the result of Poly (Neu5Acα2-3LacNAcβ-p-animophenyl/γ-PGA) and shows the result of Poly (Neu5Acα2-6LacNAcβ-p-animophenyl/α-PGA) and shows the result of Poly (Neu5Acα2-3LacNAcβ-p-animophenyl/α-PGA).

FIG. 12 shows an NMR chart for Poly (Neu5Acα2-3LacNAcβ-p-animophenyl/α-PGA).

FIG. 13 shows an NMR chart for Poly (Neu5Acα2-6LacNAcβ-p-animophenyl/60-PGA).

FIG. 14 shows an NMR chart for Poly (LacNAcβ-p-animophenyl/γ-PGA).

FIG. 15 shows an NMR chart for Poly (Neu5Acα2-3LacNAcβ-p-animophenyl/γ-PGA).

FIG. 16 shows an NMR chart for Poly (Neu5Acα2-6LacNAcβ-p-animophenyl/γ-PGA).

FIG. 17 shows an NMR chart for Poly (5-animopentyl β-lactoside/γ-PGA).

FIG. 18 shows an NMR chart for Poly (5-animopentyl β-N-acetyllactosaminide/γ-PGA).

FIG. 19 shows an NMR chart for Poly (Neu5Acα2-3Lac p-5-animopentyl/γ-PGA).

FIG. 20 shows an NMR chart for Poly (Neu5Acα2-6Lac β-5-animopentyl/γ-PGA).

FIG. 21 shows an NMR chart for Poly (Neu5Acα2-3LacNAc β-5-animopentyl/γ-PGA).

FIG. 22 shows an NMR chart for Poly (Neu5Acα2-6LacNAc β-5-animopentyl/γ-PGA).

### DETAILED DESCRIPTION OF THE PREFERRED EMBODIMENTS

Below, the present invention will be explained in detail in the following order, (1) a new polymer with sialo-oligosaccharide, (2) a method for manufacturing the polymer with sialo-oligosaccharide, (3) a reagent and a kit in which the polymer with sialo-oligosaccharide is immobilized to a support, (4) a method for determining the recognition specificity of a virus for a receptor sugar chain.

(1) New Polymer with Sialo-Oligosaccharide

As a polymer with sialo-oligosaccharide which can be used in the determining method of the present invention, the following new polymers with sialo-oligosaccharide can also be used as well as common polymers with sialo-oligosaccharide. It is much cheaper to prepare this new polymer with sialo-oligosaccharide than a common polymer and because it includes a structure which resembles a natural mucin the new polymer with sialo-oligosaccharide is suitable for the determining method of the present invention.

It is possible to exemplify the new polymer in the formulas (I), (III), (V) and (VII) below as specific examples of such a new polymer with sialo-oligosaccharide. In the polymer with sialo-oligosaccharide, sialo-oligosaccharide-substituted glutamic acid residues and non-substituted glutamic acid residues are mixed at an arbitrary ratio and this ratio is shown as a Degree of Substitution (DS) of sugar residues.

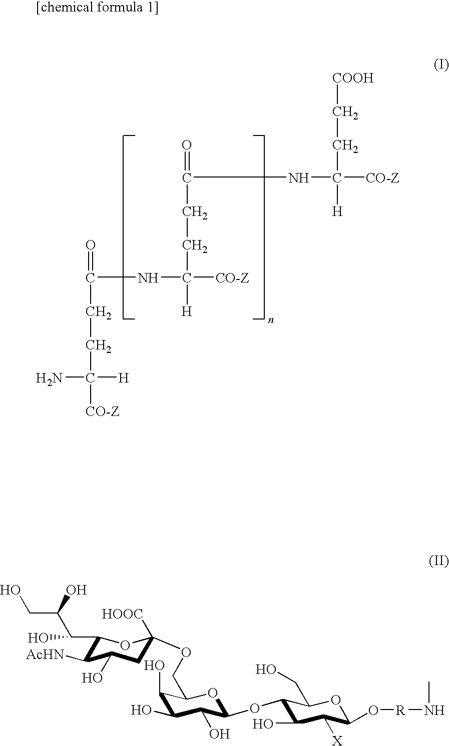

(In the formula (I), Z is a hydroxyl group or a sialo-oligosaccharide binding site expressed in the formula (II), n indicates an integer of 10 or more. In the formula (II), Ac is an acetyl group, X is a hydroxyl group or an acetyl amino group and R indicates a hydrocarbon).

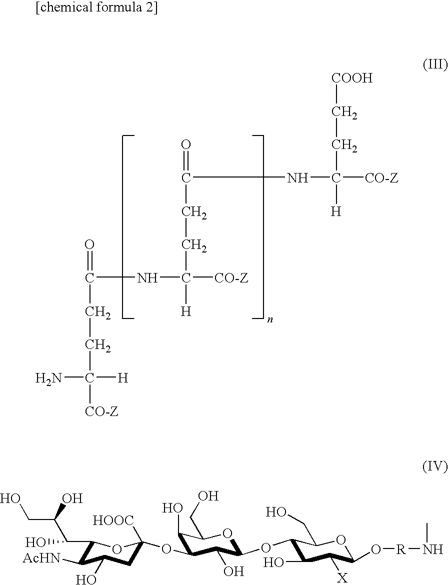

(In the formula (III), Z is a hydroxyl group or a sialo-oligosaccharide binding site expressed in the formula (IV), n indicates an integer of 10 or more. In the formula (IV), Ac is an acetyl group, X is a hydroxyl group or an acetyl amino group and R indicates a hydrocarbon).

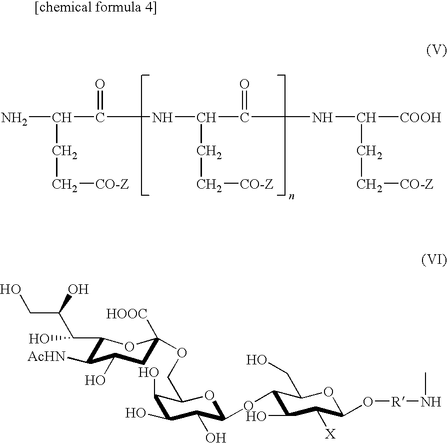

(In the formula (V), Z is a hydroxyl group or a sialo-oligosaccharide binding site expressed in the formula (VI), n indicates an integer of 10 or more. In the formula (VI), Ac is an acetyl group, X is a hydroxyl group or an acetyl amino group and R′ indicates a hydrocarbon except for phenylene).

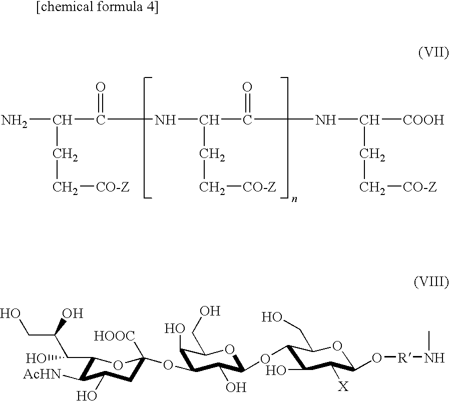

(In the formula (VII), Z is a hydroxyl group or a sialo-oligosaccharide binding site expressed in the formula (VIII), n indicates an integer of 10 or more. In the formula (VIII), Ac is an acetyl group, X is a hydroxyl group or an acetyl amino group and R′ indicates a hydrocarbon except for phenylene).

A hydrocarbon with a carbon number between 1 and 20 is preferred as the hydrocarbon expressed as R or R′ in the formula, and the hydrocarbon can be either a saturated hydrocarbon group or an unsaturated hydrocarbon group. Specifically, an alkyl group, alkenyl group, alkynyl group, cycloalkyl group, aryl group, aralkyl group, and a cycloalkyl-substituted alkyl group, and so on can be used.

Here, a linear or branched group with a carbon number between 1 and 20 can be used as an alkyl group, alkenyl group, and alkynyl group. As specific examples of an alkyl group, linear alkyl groups such a methyl group, ethyl group, n-propyl group, n-butyl group, n-pentyl group, n-hexyl group, n-octyl group, n-decyl group, n-dodecyl group and an n-tetradecyl group; branched alkyl groups such as an isopropyl group, isobutyl group, t-butyl group and 2-ethylhexyl group.

As specific examples of an alkenyl group, a vinyl group, propenyl group and allyl group can be used. As specific examples of an alkynyl group, an ethynyl group, propynyl group and a butynyl group can be used. As a cycloalkyl group, those with a carbon number between 3 and 10 and more preferably between 3 and 8, for example, a cyclopropyl group, cyclopentyl group and a cyclohexyl group can be used.

As an aryl group, those with a carbon number between 6 and 14, for example, phenyl group, tolyl group and naphthyl group can be used. As an aralkyl group, aralkyl groups with a carbon number between 7 and 14, specifically, benzyl group, phenethyl group can be used. As a cycloalkyl-substituted alkyl group, C3-C8 cycloalkyl-substituted C1-C10 alkyl groups, for example, cyclopropylmethyl group, cyclopentylmethyl group, cyclohexylmethyl group, cyclopropylethyl group, cyclopentyl ethyl group, cyclohexylethyl group, cyclopropylpropyl group, cyclopentylpropyl group, and cyclohexylpropyl group can be used.

In addition, this hydrocarbon may include a substitution group. Groups such as hydroxyl group, azide group, cyano group, alkoxy group, cycloalkyloxy group, aryloxy group and carboxyl group may be used as this substitution group. A carboxyl group may also be esterified.

The polymer with sialo-oligosaccharide of the present invention may also be a salt type or a free acid type. As a salt type, for example, alkali metal salts (for example, sodium salt, potassium salt); alkaline earth metal salts (for example, calcium salt, magnesium salt); and organic base salts (for example, trimethylamine salt, triethylamine salt, pyridine salt, picoline salt, dicyclohexylamine salt) can be used. In addition, it may also be a hydrate or a solvate such as alcohol.

In addition, the molecular weight of the polymer with sialo-oligosaccharide of the present invention is, for example, in a range between 2000 and 5,000,000. The degree of polymerization in glutamic acid units (n) is in a range, for example, between 10 and 10,000. The introduction rate of sialyl oligosaccharides to glutamic acid residues is between 10% and 80%.

The following compounds are given as specific compound examples of this type of polymer with sialo-oligosaccharide.

Poly (Neu5Acα2-6LacNAc β-5-animopentyl/γ-PGA).
Poly (Neu5Acα2-3LacNAc β-5-animopentyl/γ-PGA)
Poly (Neu5Acα2-6LacNAc β-5-animopentyl/α-PGA)
Poly (Neu5Acα2-3LacNAc β-5-animopentyl/α-PGA)
Poly (Neu5Acα2-6LacNAc β-p-animopentyl/γ-PGA)
Poly (Neu5Acα2-3LacNAc β-p-animopentyl/γ-PGA)
Poly (Neu5Acα2-6Lac β-5-animopentyl/γ-PGA)
Poly (Neu5Acα2-3Lac β-5-animopentyl/γ-PGA)
Poly (Neu5Acα2-6Lac β-5-animopentyl/α-PGA)
Poly (Neu5Acα2-3Lac β-5-animopentyl/α-PGA)
Poly (Neu5Acα2-6Lac β-p-animopentyl/γ-PGA)
Poly (Neu5Acα2-3Lac β-p-animopentyl/γ-PGA)
(PGA: polyglutamic acid, Neu5Ac: sialic acid. LacNAc: N-acetyl-lactosamine, Lac: lactose)

(2) Manufacturing Method of a Polymer with Sialo-Oligosaccharide.

In order to prepare a polymer with sialo-oligosaccharide in large amounts and at low cost, it is desirable that the enzymes which are used be unpurified products. However, in order to use these types of crude enzymes in a synthesis reaction it is not desirable to use an oligosaccharide which has been coupled with a polyglutamic acid as a reactive substrate. Therefore, after synthesizing the sialo-oligosaccharide (sialyl oligosaccharide) it is desirable to couple the sialo-oligosaccharide with the polyglutamic acid at the final step. In addition, compared to enzymes from animals, enzymes from microorganisms are easily produced in large quantities using Escherchia coli for example as hosts. However, in the case of using a glycosyltransferase derived from microorganisms, there are many cases in which it is not possible to use a glycopeptide or a glycoprotein as a sugar acceptor. As a result, in this point, it is desirable to couple the polyglutamic acid after synthezising the sialyl oligosaccharide.

Therefore, the manufacturing method of the polymer with sialo-oligosaccharide of the present invention includes the following processes.

A process (1) wherein a desired sialo-oligosaccharide including a sialic acid in a non reducing terminal is synthesized using a glycosyltransferase;

A process (2) wherein the sialo-oligosaccharide synthesized in process (1) is chemically coupled with a carboxyl group side chain of a polyglutamic acid.

A process (3) wherein a desired polymer with sialo-oligosaccharide is obtained by isolating and purifying the polymer with sialo-oligosaccharide synthesized in process (2).

### (Process 1)

Process 1 is a process wherein the desired sialo-oligosaccharide is synthesized by adding a suitable glycosyltransferase to a reaction system which contains a sugar acceptor (for example, sugar-para-nitrophenol, 5-aminoalkylated sugar) and a glycosyl donor (each variety of sugar-nucleotide).

As a glycosyltransferase which is added to the reaction system, one having an activity which shifts a sugar residue of a sugar-nucleotide to a sugar acceptor can be used, for example, galactosyltransferase, glucosyltransferase, fucosyltransferase, mannosyltransferase, and sialyltransferase can be used.

These enzymes can be any form as long as they contain the desired enzyme activity. In order to improve the ease of preparing an enzyme as well as preparation efficiency, the enzyme is preferably obtained by using an enzyme preparation technology called recombinant DNA technology in which the enzyme gene is cloned and highly expressed within the cell of a microorganism to prepare a large amount of the enzymes.

As an enzyme sample, specifically it is possible to exemplify an enzyme preparation obtained from microbial cells, treated cells or the like. It is possible to prepare the microbial cells by a method in which microorganisms are cultivated by a common method with a medium in which they can grow and are gathered by centrifugal separation or the like. Specifically, when explained using a bacterium which belongs to Escherichia coli as an example it is possible to use a bouillon medium, an LB medium (1% triptone, 0.5% yeast extract, 1% common salt) or 2×YT medium (1.6% triptone, 1% yeast extract, 0.5% common salt). After inoculating a seed cell into the medium, it is cultivated while stirring according to necessity for about 10 to 50 hours at a temperature between 30 and 50 degrees C., the cultivated solution which is obtained is separated by centrifugation, and by gathering the microorganism cells it is possible to prepare microbial cells having a desired enzyme activity.

As treated cells of a microorganism, it is possible to exemplify destroyed cells or altered cell walls or cell membranes obtained by treating the cells according to a general treatment method. As a general treatment method of cells, mechanical destruction (by using for example, a waring blender, French press, homogenizer, mortar, and the like), freezing and thawing, autolysis, drying (by for example, lyphilization, air drying, and the like), enzyme treatment (by using lysozyme and the like), ultrasonic treatment, and chemical treatment (by for example, acid, alkaline treatment, and the like), can be used.

As an enzyme preparation, a crude enzyme or a purified enzyme obtained from the above stated treated cells can be exemplified. The crude enzyme or the purified enzyme can be obtained by performing a common enzyme refining means (for example, salting-out treatment, isoelectric focusing sedimentation treatment, organic solvent sedimentation treatment, dialysis treatment and various chromatography treatments, and the like) on a fraction having the enzyme activity obtained from the above stated treated cells.

It is possible to use a commercially available sugar nucleotide and sugar acceptor. The usage concentration can be suitably set between 1 and 200 mM or more preferably in a range between 5 and 50 mM. Furthermore, in the case of using 5-amino alkylated sugar as a sugar acceptor, it is possible to amino alkylate the hydroxyl group of a sugar by utilizing a reverse reaction of a cellulase.

Synthesis of the sialo-oligosaccharide can be carried out by adding a glycosyltransferase of about 0.001 unit/ml or more or more preferably 0.01 to 10 unit/ml to a reaction system containing the above stated sugar acceptor and sugar nucleotide and reacting by stirring according to necessity between 5 and 50 degrees C. or more preferably between 10 and 40 degrees C. for about 1 to 100 hours.

The sialo-oligosaccharide which is prepared in this way can be isolated and purified by using a common separation and purification method for oligosaccharide. For example, the sialo-oligosaccharide can be isolated and purified by suitably combining reverse phase column chromatography method or ion exchange column chromatography method and the like.

### (Process 2)

Process 2 is a process for chemically coupling the sialo-oligosaccharide synthesized in process 1 to a carboxyl group side chain of a polyglutamic acid.

After a nitro group is reduced and converted to an amino group in the case where a sugar acceptor containing p-nitrophenyl is used as a acceptor in Process 1, or after a protecting group of an amino group is deprotected by a common method in the case where a 5-amino alkylated sugar is used as a sugar acceptor in Process 1, and then a polyglutamic acid is treated with a condensing agent in the presence of a base such as triethylamine or tributylamine and the like, so that a polymer with sialo-oligosaccharide is prepared.

A condition which is commonly applicable to a reduction of an aromatic nitro group can be used as a condition for a reduction reaction of a p-nitrophenyl group. As a specific example, it is possible to perform by treating it with palladium carbon in the presence of a hydrogen donor such as a hydrogen, a formic acid, an ammonium formate or a cyclohexene within water or an organic solvent such as methanol or ethanol.

The polyglutamic acid which is used as a polymer material may be either α-type or γ-type.

The coupling process can be performed by treating the polymer material with an active esterifying agent (such as, p-nitrophenylchloroformate, disuccinimidyl carbonate, or carbonyldiimidazole) for carboxyl group in the presence of a base (such as triethylamine or trimethylamine) within an organic solvent (such as dimethylformamide or dimethylsulfoxide) and then reacting with a 5-amino alkylated sugar or the product of the above stated reduction reaction.

The amount used of the 5-amino alkylated sugar or the product of the above stated reduced reaction may be dependent on the sugar substitution rate of the desired polymer with sialo sugar chain and the amount used usually may be 0.1 or more equivalent weight to 1 unit of glutamic acid of the polyglutamic acid. In addition, the amount used of a base used in the coupling reaction may be 1 or more equivalent weight to 1 unit glutamic acid of the polyglutamic acid.

The coupling reaction can be performed between −10 and 100 degrees C. In addition, a general catalyst for an acylating reaction such as 4-N,N-dimethylaminopyridine or 1-hydroxy-1H-benzotriazole may also be added according to necessity.

### (Process 3)

Process 3 is a process wherein a desired polymer with sialo-oligosaccharide is obtained by isolating and purifying the polymer with sialo-oligosaccharide synthesized in process (2). The isolating and purifying process of the polymer with sialo-oligosaccharide synthesized in process (2) may usually be performed by a method which is commonly used in purifying a protein, for example, it can be isolated and purified by suitably combining dialysis or gel filtration.

(3) a Reagent in which the Polymer with Sialo-Oligosaccharide is immobilized to a support.

There are no particular restrictions for a support for immobilizing the polymer with sialo-oligosaccharide, for example, a plate or a particle can be used. For example, a plate having well(s) (for example, a microtiter plate), or a silica gel plate used in thin-layer chromatography can be used as this plate. For example, beads or chips can be used for the particle. There are no particular restrictions for a support material, various paper, synthetic resins, metals, ceramics or glass can be used. Among these, a plate having well(s) (for example, Corning-Costar, Lab coat 2503, Cambridge Mass.) in which a polymer with sialo-oligosaccharide can be immobilized to a support by ultraviolet ray irradiation, is particularly preferred.

The sialo-oligosaccharide in the polymer with sialo-oligosaccharide can be, for example, sialyllacto-series type I sugar chain (SAα2-6(3)Galβ1-3GlcNAcβ1-), sialyllacto-series type II sugar chain (SAα2-6(3)Galβ1-4GlcNAcβ1-), sialylganglio-series sugar chain (SAα2-6(3)Galβ1-3GalNAcβ1-), and sialyl lactose sugar chain (SAα2-6(3)Gal1-4Glc-). Among these, sialyllacto-series type I sugar chain (SAα2-6(3)Galβ1-3GlcNAcβ1-) and sialyllacto-series type II sugar chain (SAα2-6(3)Galβ1-4GlcNAcβ1-) are preferred. In addition, in the polymer with sialo-oligosaccharide of the present invention, the sialic acid may be a sialic acid derivative. Furthermore, “SA” or “Neu5Ac” indicated “sialic acid (N-acetylneuraminic acid)”.

In the above stated sialo-oligosaccharide, the coupling mode of the sialic acid at the end can be, for example, “SAα2-3Galβ1-” (below referred to as (2-3 type)), “SAα2-6Galβ1-” (below referred to as (2-6 type)) and “SAα2-8Galβ1-” (below referred to as (2-8 type)).

The above stated polymer in the polymer with sialo-oligosaccharide is not particularly limited, for example, a chemically synthesized polymer such as polyglutamic acid, polyacrylamide and polystyrene, a natural glycoprotein such as fetuin, as well as a lipid with sugar chain and the like, can be used. As the lipid with sugar chain, a chemical synthetic glycolipid having a lipid moiety which is a fatty acid or its derivative, a natural ganglioside or glycolipid such as sialyparagloboside, sialylactotetraosylceramide and the like, as well as a chemical synthetic ganglioside or glycolipid and the like can be used. Among these, polyglutamic acid is particularly preferred and may be either α-type or γ-type.

As a specific example of the polymer with sialo-oligosaccharide, there is a polyglutamic acid with sialo-oligosaccharide obtained by introducing a sialyl oligosaccharide to the polyglutamic acid. Its molecular weight is, for example, in a range between 2,000 and 5,000,000 and the degree of polymerization in glutamic acid units is in a range, for example, between 10 and 10,000 and the introduction rate of sialyl oligosaccharides to glutamic acid residues is between 10% and 80%. As a polyglutamic acid with sialo-oligosaccharide obtained by introducing a sialyl oligosaccharide to the polyglutamic acid, apart from the already stated new polyglutamic acid with sialo-oligosaccharide there are other known polymers with sialo-oligosaccharides which are outlined below.

(2-3 type)

### Poly [para-aminophenyl (N-acetylneuraminyl-(2-3)-N-acetyl)-β-lactosaminide)-L-glutamine-co-glutamic acid] [Poly (Neu5Acα2-3Galβ1-4GlcNAcβ-pAP/Gln-co-Glu)]

(2-6 type)

### Poly [para-aminophenyl (N-acetylneuraminyl-(2-6)-N-acetyl)-β-lactosaminide)-L-glutamine-co-glutamic acid][Poly (Neu5Acα2-6Galβ1-4GlcNAcβ3-pAP/Gln-co-Glu)]

This type of polyglutamic acid with sialo-oligosaccharide can be prepared by known methods other than the manufacturing methods of the present invention stated above. Specifically, it is possible to prepare this type of polyglutamic acid with sialo-oligosaccharide by introducing paranitrophenyl glycosides (para-nitrophenylt N-acetyl-β-lactosaminide) which are synthesized by a glycotransfer reaction of β-galactosidase to polyglutamic acids, and further sialylating the introduced oligosaccharides using α2,3-(N)- and α2,6-N-sialytransferase. A specific example of this preparation method will be explained as a reference example. As a method for synthesizing a paranitrophenyl glycoside by a glycotransfer reaction of β-galactosidase, the method in T. Usui et al. (Carbohydr Res), Vol. 244, pp. 315 to 323 [1993] can be used. As a method for introducing paranitrophenyl glycosides to polyglutamic acids, the method in X. Zeng et al. (Carbohyd Res), Vol. 312, pp. 209 to 217 [1998] can be used. As a method for sialylating an oligosaccharide, the method in X. Zeng et al. (Arch. Biochem. Biophys.) Vol. 383, pp. 28 to 37 [2000] can be used.

Immobilization of a polymer with sialo-oligosaccharide to a support can be performed using hydrophobic bonding, ion binding and covalent binding and the like. For example, in the case of immobilizing a polyglutamic acid with sialo-oligosaccharide to a synthetic resin plate (for example, a microtiter plate) having well(s), ultraviolet ray irradiation has the greatest effect and is an easy method.

Here, in the case of immobilizing a certain specific substance to the support, a method is commonly used in which a solution including this substance is brought into contact with the support and after removing the solution, irradiation of ultraviolet rays is performed. However, the inventors of the present invention have found that with this method, the polymer with sialo-oligosaccharide cannot be immobilized to the support. In addition, in order to solve this problem, a series of research was continued at which point it was found that it is possible to immobilize a polymer with sialo-oligosaccharide to the surface of a support by bringing a solution which contains the polymer with sialo-oligosaccharide into contact with the support and while in this state irradiating with ultraviolet rays and subsequently removing the solution.

Specifically, a solution containing a polyglutamic acid with sialo-oligosaccharide is brought into contact with a plate and while in this state the support is irradiated with ultraviolet rays. Following this, it is possible to immobilize the polyglutamic acid with sialo-oligosaccharide to the surface of the support by removal of the solution. Furthermore, during the ultraviolet ray irradiation treatment, because reaction times will differ due to the strength of the ultraviolet rays and distance to the plate, it is preferable to set these conditions in advance.

In order to protect against nonspecific adsorption of a virus it is preferred that the support to which the prepared polymer with sialo-oligosaccharide is immobilized is treated with blocking. This blocking treatment can be performed by using, for example, bovine serum albumin (BSA), delipidated BSA, egg albumin, casein or a commercially available blocking agent and the like.

(4) a Method and a Kit for Determining the Recognition Specificity of a Virus for a Receptor Sugar Chain.

In the determination method of the present invention, the assay of the binding degree can be performed in accordance with an immunologic assay method such as ELISA method, immunochromatography or immune agglutination method. For example, in order to assay a higher sensitivity, it is possible to exemplify a suitable example of an assay by a sandwich immunologic assay. In the sandwich immunologic assay, an antivirus primary antibody against a virus and a labeled secondary antibody or a labeled protein A against the antivirus primary antibody may be used. However, this is not limited to the sandwich immunologic assay, it is possible to assay the binding degree by the degree of agglutination by using a particle support such as beads as the support. Furthermore, it is clear that detection methods of specific components of viruses (for example, detection of hemagglutinin and neuraminidase which are spike proteins of viruses, and detection of their bioactivity) by methods other than an immunologic assay can also be used.

When the above stated sandwich immunologic assay is explained in more detail, the antivirus primary antibody is not particularly limited, a polyclonal antibody and a monoclonal antibody may be used. As the polyclonal antibody, for example, there is an anti influenza virus rabbit serum. In addition, as the monoclonal antibody, there is an antibody which reacts to all A viruses, such as a monoclonal antibody against nucleoproteins of A viruses. Furthermore, the origin of the antibody is not particularly limited, for example, rabbit antibody, mouse antibody, rat antibody, goat antibody, dog antibody or sheep antibody can be used. The class of the antibody is also not particularly limited, IgG, IgM, IgA, IgD, and IgE can all be applied.

The label of the above stated labeled secondary antibody or the labeled protein A, is not particularly limited, for example, enzyme label (for example, horseradish peroxidase), fluorescent label and radioactive label and the like can be used. Furthermore, the origin of the antibody is not particularly limited, for example, rabbit antibody, mouse antibody, rat antibody, goat antibody, dog antibody or sheep antibody can be used. The class of the antibody is also not particularly limited, IgG, IgM, IgA, IgD, and IgE can all be applied. As the labeled secondary antibody, a rabbit IgG antibody labeled with enzyme is preferred.

In the present invention, the virus to be determined is not particularly limited. A variety of viruses can be applied according to the polymer with sialo-oligosaccharide to be used. For example, influenza virus, paramyxovirus group, parainfluenza group, rotavirus, adenovirus, coronavirus, polyomavirus group and the like can be applied. As the influenza virus, highly pathogenic avian influenza A virus, human influenza A virus and human influenza B virus and the like can be applied.

A virus sample which is used in an assay may be a virus sample which has been inactivated treated. For example, a virus incubated chicken chorioallantois solution inactivated by ether treatment can be assayed just as it is without being concentrated by the method of the present invention.

The assay procedure itself may be performed according to a known means of the methods which is adopted. For example, in the case where an immunologic assay is applied, an immobilized polymer with sialo-oligosaccharide is made to react with a virus sample, and after BF separation according to necessity, it is further made to react with a labeled antibody (two step method) or a solid antibody, a sample to be examined and a labeled antibody are made to react simultaneously (one step method). Then, it is possible to detect the recognition specificity of a virus for a receptor sugar within the sample by a later step of a known method itself.

Furthermore, the details of the immunologic assay may be referenced in, for example, the following documents.

(1) Edited by Irie Hiroshi [Sequel of Radioimmunoassay] (Kodansha Ltd. Published 1979, May 1st)
(2) Edited by Ishikawa Eiji et al. [Enzyme-Linked Immunosorbent Assay] (Second edition) Igaku Shoin Ltd. Published 1982, Dec. 15th)
(3) The Japanese Journal of Clinical Pathology Extra Edition Special featuring No. 53 (Immunoassay for Clinical examination—technology and application—) (The Clinical Pathology Press, 1983)
(4) “Biotechnology encyclopedia” (CMC. Ltd, 1986, Oct. 9th)

### (5) [Methods in ENZYMOLOGY Vol. 70]

(Immunochemical techniques (Part A))

### (6) [Methods in ENZYMOLOGY Vol. 73]

(Immunochemical techniques (Part B))

### (7) [Methods in ENZYMOLOGY Vol. 74]

(Immunochemical techniques (Part C))

### (8) [Methods in ENZYMOLOGY Vol. 84]

(Immunochemical techniques (Part D: Selected Immunoassay))

### (9) [Methods in ENZYMOLOGY Vol. 92]

(Immunochemical Techniques (Part E: Monoclonal Antibodies and General Immunoassay Methods))

### [(5) to (9) Published by Academic Press]

While the highly pathogenic avian influenza A virus strongly recognizes the 2-3 type sialo-oligosaccharide, its recognition, coupling or affinity properties towards the 2-6 type sialo-oligosaccharide are weak. Alternatively, the human influenza A virus and the human influenza B virus strongly recognize the 2-6 type sialo-oligosaccharide but their recognition, coupling or affinity properties towards the 2-3 type sialo-oligosaccharide are weak. Therefore, in the method of the present invention, the polymers with sialo-oligosaccharide of both the 2-3 type and 2-6 type are used, the binding degrees to each polymer with sialo-oligosaccharide are assayed and by comparing these it is possible to determine the avian infecting influenza virus and the human infecting influenza virus.

In addition, in the determining method of the present invention, a support may be used wherein two or more polymers with sialo-oligosaccharide are immobilized on the surface of the support. In this case, by bringing a sample of a virus into contact with each of the polymers with sialo-oligosaccharide and assaying the binding degree therein it is possible to determine the recognition specificity of a virus for a receptor sugar chain, that is, the infection type of the virus, and detect a change in a host infected caused by a virus mutation by comparing the results. That is, a plate containing a plurality of wells, in which a polymer with sialo-oligosaccharide selected among different types is immobilized to each well or each line is used. Then, the virus is applied on each well, and by comparing the recognition specificity of each well, the infection type of the virus and a change in a host infected cause by a mutation is determined. Apart from this, for example, pluralities of supports are used in which a different kind of polymer with sialo-oligosaccharide is immobilized to each support. In this way, the binding degree of a virus is assayed for each support which is bound with one of the two or more kinds of polymer with sialo-oligosaccharide, the results are compared and a virus infection type and a change in an infected host due to a mutation is detected. In this case, as stated above, it is possible to use a particle support such as beads as a support, a virus may be supplied to each support, and a virus infection type may be determined by comparing the recognition specificity between particle supports by for example, the degree of agglutination.

Next, in addition to the support which immobilizes the polymer with sialo-oligosaccharide, it is preferred that the kit of the present invention further includes an antivirus antibody (for example, an antivirus primary antibody for a virus and a labeled secondary antibody or a labeled protein A for the antivirus primary antibody) for detecting a virus which is trapped by the support. The antibody is stated above.

### Examples

Next, examples of the present invention will be explained. Furthermore, the present invention is not limited by the following examples.

### <HPLC>

All the samples were analyzed after being filtrated by a filter of 0.45 μl. The following conditions were used in the analysis.

### Column: Mightysil Si60 (ø 4.6×250 mm)

Column temperature: 40 degrees C.
Flow rate: 1.0 ml/min
Detection wave length: 210 nm

### Solvent: 90% CH₃CN

### Or;

### Column: YMC Pro C18RS (ø6.0×150 mm)

Column temperature: 40 degrees C.
Flow rate: 1.0 ml/min
Detection wave length: 300 nm

### Solvent: 20% MeOH-50 mM TEAA

<NMR>

Analysis Apparatus: JEOL EX-270 NMR spectrometer,

JEOL lamda 500FT NMR spectrometer

Bruker AV-500 NMR spectrometer

External standard: TPS [sodium3-(trimethysilyl)-propionate]

### Solvent: D₂O

Temperature: 25 degrees C. or 60 degrees C.
Sample tube: ø3 or 5 mm

(Abbreviations)

pNP: p-nitrophenol

### Lac: Lactose (Galβ1-4Glc)

### LacNAc: N-acetyllactosamine (Galβ1-4GlcNAc)

Neu5Ac: N-acetylneuraminic acid
CMP-NeuAc: CMP-N-acetylneuraminic acid
γ-PGA: γ-polyglutamic acids
BOP: Benzotriazol-1-yloxytris-(dimethylamino) phosphonium hexafluorophosphate
HOBt: 1-Hydroxybenzotriazole hydrate
PBS: 10 mM Phosphate buffered saline (pH 7.4)
TPS: Sodium 3-(trimethylsilyl)-propionate
DP: Degree of polymerization (degree of polymerization of
γ-polyglutamic acid)
DS: Degree of substitution (degree of sugar residue substitution % in the case where DP is 100%)

### IPTG: Isopropyl-beta-D-thiogalactopyranoside

EDTA: Ethylenediaminetetracetic acid
dATP: 2′-deoxyadenosine 5′-triphospate
dGTP: 2′-deoxyguanosine 5′-triphospate
dCTP: 2′-deoxycytidine 5′-triphospate
dTTP: 2′-deoxythymidine 5′-triphospate

### Pd—C: Palladium on Carbon

### DMF: Dimenthylformamide

### Et₃N: Triethyamine

pNPCF: para-Nitrophenyl chloroformate
DMAP: N,N-dimethyl-4-aminopryridin
DMSO: Dimethyl sulfoxide

### AP: Alkaline Phosphatase

### Example 1

### Preparation of 3′-SLN-α PGA (Poly(Neu5Ac α 2-3LacNAcβ-p-aminophenyl/α-PGA)) and 6′-SLN-α PGA (Poly(Neu5Ac α 2-6LacNAcβ-p-aminophenyl/α-PGA))

3′-SLN-α PGA and 6′-SLN-α PGA were prepared on the synthesis path shown in the formula (IX).

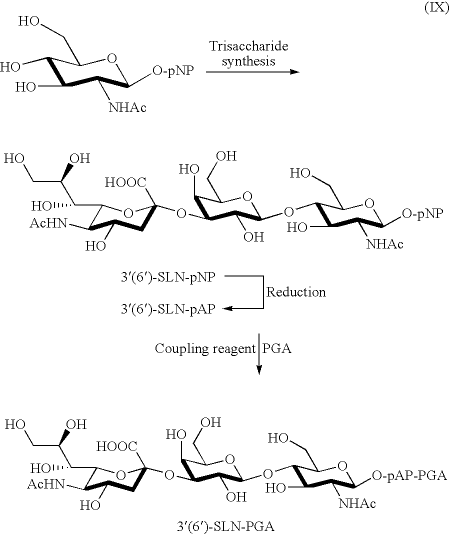

### (1) Preparation of β1,4-Galactosyltransferase (β1,4-GalT)

Preparation of β1,4-GalT was performed using the expression plasmid pTGF-A cited in the method by Noguchi et al. (Patent Document 2002-335988). Escherichia coli JM109 which holds the pGTF-A was inoculated in 50 ml of 2×YT medium which contained 100 μg/ml of ampicillin, and was shaken at 30 degrees C. and cultivated. At the point where the cell density reached 4×10⁸ cells/ml, IPTG was added so that the cultivated solution became a final concentration of 0.1 mM and cultivation was continued by further shaking for 16 hours at 30 degrees C. After cultivation had finished, the cells were collected by centrifugal separation (9000×g, 20 minutes) and suspended in 5 ml of a buffer solution (10 mM tris-HCl (pH 8.0), 1 mM EDTA). An ultrasonic wave treatment was performed and the cells were crushed. The cell residues were removed by further centrifugal separation (20,000×g, 10 minutes) and the supernatant fraction which was obtained was used as an enzyme solution. The activity of β1,4-GalT in the enzyme solution was assayed using the method cited in Patent Document 2005-335988.

### (2) Preparation of α2,3-Sialyltransferase (α2,3-SiaT)

Preparation of α2,3-SiaT was performed using the expression plasmid pMaI-siaT cited in the method by Noguchi et al. (Patent Document 2002-335988). Escherichia coli JM109 which holds the pMaI-siaT was inoculated in 50 mL of 2×YT medium which contained 100 μg/ml of ampicillin, and was shaken at 30 degrees C. and cultivated. At the point where the cell density reached 4×10⁸ cells/ml, IPTG was added so that the cultivated solution became a final concentration of 0.1 mM and cultivation was continued by further shaking for 16 hours at 30 degrees C. After cultivation had finished, the cells were collected by centrifugal separation (9000×g, 20 minutes) and suspended in 5 ml of a buffer solution (100 mM tris-HCl (pH 8.0), 10 mM MgCl). An ultrasonic wave treatment was performed and the cells were crushed. The cell residues were removed by further centrifugal separation (20,000×g, 10 minutes) and the supernatant fraction which was obtained was used as an enzyme solution. The activity of α2,3-SiaT in the enzyme solution was assayed using the method cited in Patent Document 2005-335988.

### (3) Preparation of α2,6-Sialyltransferase (α2,6-SiaT)

Chromosomal DNA from Photobacterium subsp. damsela (NBRC No. 15633 or ATCC 33539) was prepared in the following procedure. First, after the lyophilized cell of the bacteria was suspended in 100 μL of 50 mM tris-HCl buffer solution (pH 8.0), containing 20 mM EDTA, 10 μL of 10% SDS solution was added and lysized by leaving to rest for 5 minutes at room temperature. Then, chromosomal DNA was prepared from the cell by dissolving a sediment which was obtained from this lysis solution by phenyl extraction and ethanol sedimentation into 20 μL of TE buffer (10 mM tris-HCl buffer (pH 8.0), 1 mM EDTA)

The prepared DNA was made into a template, and two kinds of primer DNA (A) and (B) shown below were synthesized according to a common method. DNA of a region which includes a bst gene (Submitted to NCBI, Accession No. AB012285) which encodes for the β-galactoside a 2,6-sialyltransferase of the Photobacterium damsela was amplified by PCR method using the two kinds of primer.

Primer (A):
5′ - GTGTGGCATAGTACGCACTT - 3′
Primer (B):
5′ - AGGTCGCCACATTTACGATG - 3′

The amplification by the PCR method of the DNA of the region which includes the bst gene was carried out by repeating 36 times a series of steps which include a thermal denaturation (94 degrees C., 1 minute), annealing (47 degrees C., 1 minute), and elongation reaction (72 degrees C., 2 minutes) using a DNA Thermal Cycler Dice (Takara Bio) with 100 μl of a reactive solvent. This reaction solution included 10 μl of 10× Pyrobest Buffer (Takara Bio), 0.2 mM dATP, 0.2 mM dGTP, 0.2 mM dCTP, 0.2 mM dTTP, 0.1 ng of the template DNA, 0.2 μM DNA primer (A) and 0.2 μM DNA primer (B) and 2.5 units of Pyrobest DNA polymerase (Takara Bio).

The DNA after amplification was separated by agarose gel electrophoresis according to a method in a document (Molecular Cloning, (Edited by Maniatis et al., Cold Spring Harbor Laboratory, Cold Spring Harbor, N.Y. (1982)) and 2.3 kb of DNA fragments were purified. This DNA was made into a template and using two kinds of primer DNA shown below (C) and (D), the bst gene of the Photobacterium damsela was amplified by the PCR method.

Primer (C):
5′ - CTTGGATCCTGTAATAGTGACAATACCAGC - 3′
Primer (D):
5′ - TAAGTCGACTTAAGCCCAGAACAGAACATC - 3′

The amplification by the PCR method of the bst gene was carried out by repeating 36 times a series of steps which include thermal denaturation (94 degrees C., 1 minute), annealing (52 degrees C., 1 minute), and elongation reaction (72 degrees C., 2 minutes) using a DNA Thermal Cycler Dice (Takara Bio) with 100 μl of a reaction solution. This reaction solution included 10 μl of 10× Pyrobest Buffer (Takara Bio), 0.2 mM dATP, 0.2 mM dGTP, 0.2 mM dCTP, 0.2 mM dTTP, 0.1 ng of the template DNA, 0.2 μM DNA primer (C) and 0.2 μM DNA primer (D) and 2.5 units of Pyrobest DNA polymerase (Takara Bio).

The DNA after amplification was separated by agarose gel electrophoresis and 1.5 kb of DNA fragments were purified. The obtained DNA fragments were digested using restriction enzymes BamHI and Sal 1, and connected to a plasmid pTrc12-6 (Patent Document 2001-103973) which was digested by the same restricted enzymes BamHI and Sal1 by use of T4DNA ligase. Escherichia coli K12 strain JM109 (obtained from Takara Bio) was transformed using a ligating solution and plasmid p12-6-pst Δ N was isolated from the obtained kanamycin resistant transformant.

Escherichia coli JM109 which held the plasmid p12-6-pst Δ N was inoculated in 100 ml of a medium (2% peptone, 1% yeast extract, 0.5% NaCl, 0.15% glucose) which contained 25 μg/ml of kanamycin, and was shaken at 30 degrees C. and cultivated. After 5 hours IPTG was added so that the cultivated solution became a final concentration of 0.2 mM and cultivation was continued by further shaking for 20 hours at 18 degrees C. After cultivation had finished, the cells were collected by centrifugal separation (9000×g, 10 minutes) and suspended in 2.5 ml of a buffer solution (20 mM sodium acetate (pH 5.5)) and a suspended solution was obtained. The suspended solution was iced and subjected to an ultrasonic wave treatment (50 W, 2 minutes, three times) using an ultrasonic homogenizer made by Branson (model 450 Sonifier), separated by centrifugal separation at 12,000×g, at 4 degrees C., and soluble fractions (supernatant) were collected.

The supernatant fraction obtained in this way was used as an enzyme sample and the activity of a 2,6-sialyltransferase in the enzyme sample was assayed. The result showed that it was 0.44 units/min/ml enzyme solution.

In addition, the activity of a 2,6-sialyltransferase is the transformation activity from CMP-NeuAc and N-acetyllactosamine to 6′-SialylLacNAc which was assayed and calculated by the method shown below. That is, the α2,6-sialyltransferase enzyme sample was added to 25 mM tris-HCl buffer solution (pH 5.5), 50 mM CMP-NeuAc, and 10 mM N-acetyllactosamine and made to react at 37 degrees C. for 10 minutes. The reaction solution was boiled for three minutes to stop the reaction and a sugar analysis was performed by HPAEC-CD (High performance anion exchange chromatography coupled with conductivity detection). A Carbopac PA1 column (4×250 mm) made by Dionex, was used in separation and concentration gradient was performed by using (A) 0.1 M NaOH solution and (B) solution of 0.1 M NaOH and 0.5 M sodium acetate (0 to 10 minutes: B=0%, 10 to 25 minutes: B=45%, 25 to 30 minutes: B=100%) as eluates. The consumed amount of LacNAc and produced amount of 6′-SialylLacNAc in the reaction solution were calculated from the HPAEC-CD analysis result and the activity which transforms to NeuAc of 1μ mole into N-acetyllactosamine at 37 degrees C. in one minute was given as one unit.

### (4) Synthesis of 3′-SLN-pNP (p-nitrophenyl-Neu5Ac α2-3 LacNAc)

75 ml of a solution which included 100 mM tris-HCl (pH 8.0), 20 mM MgCl₂, 20 mM GlcNAc-pNP ((p-nitrophenyl-GlcNAc), 30 mM UDP-Gal, 5.0% (v/v) Acetonitrile, and 0.1 U/ml β1,4-GalT, was incubated for 6 hours at 37 degrees C. 20 mM MnCl₂, 30 mM CMP-NeuAc, 1 U/ml alkaline phosphatase (Takara Bio), and 0.22 U/ml α2-3-SiaT were added to this reaction solution and made 100 ml. After the reaction solution was incubated for 20 hours at 37 degrees C., it was boiled for 5 minutes, divided using centrifugal separation (8000 rpm, 20 minutes) and supernatants were collected.

The synthesized solution was applied on an ODS column (340 mL, equilibrated by 50 mM triethylamine hydrogencarbonate), and the desired substance was eluted with 5 to 10% MeOH-50 mM triethylamine hydrogencarbonate. 3′-SLN-pNP containing fractions were collected and after the collected fractions were concentrated, they were azeotropically boiled with water five times and the triethylamine hydrogencarbonate was removed. The solution collected from the ODS column was made to be 150 mL, stuck on a DEAE column (330 mL), eluted with 0.05 N ammonium hydrogencarbonate water solution and the 3′-SLN-pNP containing fractions were collected. This was then concentrated and further azeotropically boiled together with water five times and the ammonium hydrogencarbonate was removed. MeOH (20 mL) was added to the residue to be treated by azeotropic dehydration. This was then dried in a vacuum (50 degrees C., 3 hours), and 963 mg of 3′-SLN-pNP (79% including the remained 0.8 molecules of MeOH) was obtained.

(NMR of the Obtained 3′-SLN-pNP)

¹H-NMR (D₂O): δ 8.26 (2H, d, J=9.3 Hz), 7.20 (2H, d, J=9.3 Hz), 5.35 (1H, d, J=8.4 Hz), 4.61 (1H, d, J=7.9 Hz), 4.16-3.59 (19H, m), 2.78 (1H, dd, J=4.6, 12.5 Hz), 2.04 (3H, s), 2.02 (3H, s), 1.82 (1H, t, J=12.2 Hz)

### (5) Synthesis of 6′-SLN-pNP (p-nitrophenyl-NeuAc α2-6 LacNAc)

75 ml of a solution which included 100 mM tris-HCl (pH 8.0), 20 mM MgC1₂, 20 mM GlcNAc-pNP, 30 mM UDP-Gal, 5.0% (v/v) Acetonitrile, and 0.1 U/ml β1,4-GalT, was incubated for 6 hours at 37 degrees C. 20 mM MnCl₂, 30 mM CMP-NeuAc, 1 U/ml of alkaline phosphatase (Takara Bio), and 0.22 U/ml α2-6-SiaT were added to this reaction solution and made 100 ml. After the reaction solution was incubated for 20 hours at 37 degrees C., it was boiled for 5 minutes, divided using centrifugal separation (8000 rpm, 20 minutes) and supernatants were collected.

The synthesized solution was applied on an ODS column (300 mL, equilibrated by 50 mM triethylamine hydrogencarbonate), and the desired substance was eluted with 5 to 10% MeOH-50 mM triethylamine hydrogencarbonate. 6′-SLN-pNP containing fractions were collected and after the collected eluted fractions were concentrated, they were azeotropically boiled together with water five times and the triethylamine hydrogencarbonate was removed. The solution collected from the ODS column was made to be 150 mL, applied on a DEAE column (300 mL), eluted with 0.05 N ammonium hydrogencarbonate water solution and the 6′-SLN-pNP eluted fractions were collected. This was then concentrated and further azeotropic boiled with water five times and the ammonium hydrogencarbonate was removed. MeOH (20 mL) was added to the residue to be treated by azeotropic dehydration. This was then dried in a vacuum (50 degrees C., 2 hours), and 1.05 g of 6′-SLN-pNP (86% including the remained 0.8 molecules of MeOH) was obtained.

(NMR of the obtained 3′-SLN-pNP)

¹H-NMR (D₂O): δ 8.26 (2H, d, J=9.3 Hz), 7.21 (2H, d, J=9.3 Hz), 5.39 (1H, d, J=8.5 Hz), 4.50 (1H, d, J=7.9 Hz), 4.10 (1H, dd, J=8.5, 10.5 Hz), 4.04-3.56 (18H, m), 2.70 (1H, dd, J=4.6, 12.4 Hz), 2.06 (3H, s), 2.04 (3H, s), 1.75 (1H, t, J=12.2 Hz)

### (6) Synthesis of 3′-SLN-pAP (p-aminophenyl-NeuAc α2-3 LacNAc)

3′-SLN-pNP (503 mg, 0.6 m mol) was dissolved in distilled water (30 mL), and 10% Pd—C (50 mg) and ammonium formate (378 mg, 6.0 mmol) were added and stirred at room temperature. After 2 hours, a HPLC analysis was performed and after confirmation that the raw material had completely disappeared, a reaction was made an open system and stirred at room temperature for 21 hours. The Pd—C was eliminated by filtration and after concentrating the filtrate, the filtrate was azeotropically boiled three times with water (3 ml)-triethylamine (1 ml×1, 0.5 ml×2) and after making 3′-SLN-pAP-Et₃N salt, was azeotropically dehydrated three times with DMF (3 mL). The residue was prepared as a 2.4 mL solution (0.25 M) of DMF.

(NMR of ammonium salt)

¹H-NMR (D₂O): δ 6.97 (2H, d, J=8.9 Hz), 6.88 (2H, d, J=8.9 Hz), 5.04 (1H, d, J=8.5 Hz), 4.60 (1H, d, J=7.9 Hz), 4.14 (1H, dd, J=3.1, 9.9 Hz), 4.04-3.58 (18H, m), 2.78 (1H, dd, J=4.6, 12.5 Hz), 2.05 (3H, s), 2.05 (3H, s), 1.82 (1H, t, J=12.2 Hz)

### (7) Synthesis of 6′-SLN-pAP (p-aminophenyl-NeuAc α2-6 LacNAc)

6′-SLN-pNP (502 mg, 0.6 m mol) was dissolved in distilled water (30 mL), and 10% Pd—C (50 mg) and ammonium formate (378 mg, 6.0 m mol) were added and stirred at room temperature. After 2.5 hours, a HPLC analysis was performed and after confirmation that the raw material had completely disappeared, a reaction was made an open system and stirred at room temperature for 21 hours. The Pd—C was eliminated by filtration and after concentrating the filtrate, the filtrate was azeotropically boiled three times together in water (3 ml)-triethylamine (1 ml×1, 0.5 ml×2) and after making 6′-SLN-pAP-Et₃N salt, was azeotropically dehydrated three times in DMF (3 mL). The residue was prepared as a 2.4 mL solution (0.25 M) of DMF.

(NMR of ammonium salt)

¹H-NMR (D₂O): δ 6.97 (2H, d, J=8.8 Hz), 6.86 (2H, d, J=8.8 Hz), 5.07 (1H, d, J=8.5 Hz), 4.48 (1H, d, J=7.9 Hz), 4.03-3.54 (19H, m), 2.69 (1H, dd, J=4.6, 12.4 Hz), 2.07 (3H, s), 2.04 (3H, s), 1.74 (1H, t, J=12.2 Hz)

### (8) Synthesis of 3′-SLN-αPGA (Poly (Neu5Ac α2-3LacNAcβ-p-aminophenyl/α-PGA))

α-PGA (13 mg, 0.1 m mol as glu unit) and Et₃N (17 μl, 0.12 m mol) were dissolved in DMF (1.0 ml), then DMAP (1.2 mg, 0.01 m mol) and pNPCF (24 mg, 0.12 m mol) were added at 0 degrees C. and stirred for 1 hour at the same temperature. A DMF solution of 3′-SLN-pAP (0.25 M, 0.4 ml, 0.1 m mol), HOBt (31 mg, 0.2 m mol) and Et₃N (14 μl, 0.1 m mol) were each added and stirred for 24 hours at room temperature. After water (200 μl) was added to the reaction solution, 1 N-NaOH (1.6 ml) was added and stirred for 1 hour at room temperature. The sedimentation that arose was eliminated by centrifugal separation (15000 rpm, 5 minutes).

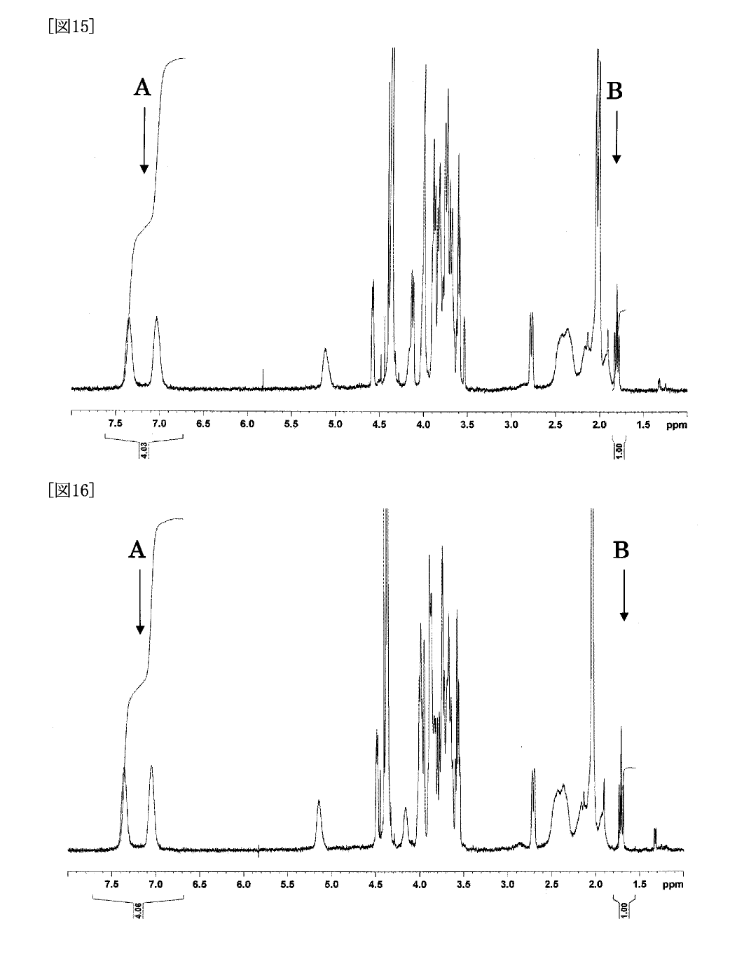

1.5 ml of supernatant was put into a dialysis tube and dialyzed against 200 ml of distilled water. A 3′-SLN-αPGA solution was collected and concentrated to 0.8 ml by an evaporator condenser (bath temperature 40 degrees C.), and applied on a gel filtration (Sephadex G-50 F, 8 ml). The sample was applied and eluted with 10 ml of ultrapure water, and the total volume was collected from the applied sample. The collected sample was put into a dialysis tube and dialyzed against 1000 ml of distilled water and ultrapure water. The dialyzed sample was collected and applied on an ion exchange column (Dowe×AG 50W-8×, 3 ml). The sample was then eluted with 30 ml of ultrapure water after being stuck and the total volume was collected from the stuck solution (40 to 45 ml). The collected solution was reduced to 0.8 ml by an evaporator condenser (bath temperature 40 degrees C.) and 37.4 mg of 3′-SLN-αPGA was obtained by lyophilization (shelf temperature 20 degrees C., one night). The obtained 3′-SLN-αPGA was analyzed by ¹H-NMR and the sugar residue substitution rate was calculated as 68% based on the formula below (see FIG. 12).

Sugar residue substitution rate (%)=(A×100)/(C−(3A/4)−4B)

(NMR of the obtained 3′-SLN-αPGA)

¹H-NMR (D₂O 60 degrees C.): δ 7.26 (brs), 6.93 (brs), 5.00 (brs), 4.57 (brs), 4.12 (d, J=9.6 Hz), 4.07-3.50 (m), 2.78 (d, J=8.2 Hz), 2.41 (brs), 2.29-1.92 (m), 1.81 (t, J=12.0 Hz)

### (9) Synthesis of 6′-SLN-αPGA (Poly (Neu5Ac α2-6LacNAcβ-p-aminophenyl/α-PGA))

α-PGA (13 mg, 0.1 m mol as glu unit) and Et₃N (17 μl, 0.12 m mol) were dissolved in DMF (1.0 ml), then DMAP (1.2 mg, 0.01 m mol) and pNPCF (24 mg, 0.12 m mol) were added at 0 degrees C. and stirred for 1 hour at the same temperature. A DMF solution of 6′-SLN-pAP (0.25 M, 0.4 ml, 0.1 m mol), HOBt (31 mg, 0.2 m mol) and ET₃N (14 μl, 0.1 m mol) were each added and stirred for 19 hours at room temperature. After water (200 μl) was added to the reaction solution, 1 N-NaOH (1.6 ml) was added and stirred for 1 hour at room temperature. The sedimentation that arose was eliminated by centrifugal separation (15000 rpm, 5 minutes).

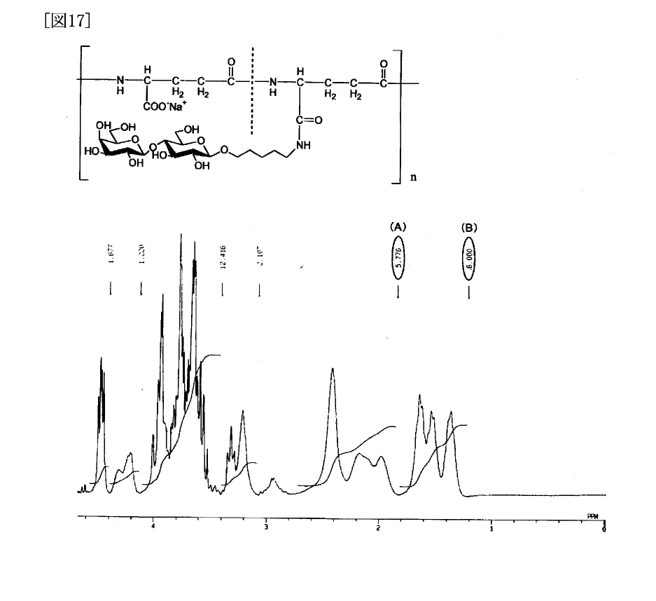

1.5 ml of supernatant was put into a dialysis tube and dialyzed against 200 ml of distilled water. A 6′-SLN-αPGA solution was collected and concentrated to 0.8 ml by an evaporator condenser (bath temperature 40 degrees C.), and applied on a gel filtration (Sephadex G—50 F, 8 ml). The sample was applied and eluted with 10 ml of ultrapure water, and the total volume of applied sample was collected. The collected sample was put into a dialysis tube and dialyzed against 1000 ml of distilled water and ultrapure water. The dialyzed sample was collected and applied on an ion exchange column (Dowe×AG 50 W-8×, 3 ml). The sample was then eluted with 30 ml of ultrapure water after being stuck and the total volume was collected from the stuck solution (40 to 45 ml). The collected solution was concentrated to 0.8 ml by an evaporator condenser (bath temperature 40 degrees C.) and 39.6 mg of 6′-SLN-αPGA was obtained by lyophilization (shelf temperature 20 degrees C., one night). The obtained 6′-SLN-αPGA was analyzed by ¹H-NMR and the sugar residue substitution rate was calculated as 66% based on the formula below (see FIG. 13).

Sugar residue substitution rate (%)=(A×100)/(C−(3A/4)−4B)

(NMR of the obtained 6′-SLN-αPGA)

¹H-NMR (D₂O 60 degrees C.): δ 7.28 (brs), 6.97 (brs), 5.06 (brs), 4.47 (d, J=7.7 Hz), 4.00-3.55 (m), 2.71 (dd, J=4.2, 12.2 Hz), 2.41 (brs), 2.29-1.90 (m), 1.71 (t, J=12.0 Hz)

### Example 2

### Preparation of 3′-SLN-γ PGA (Poly(Neu5Ac α2-3LacNAcβ-p-aminophenyl/γ-PGA)) and 6′-SLN-γPGA (Poly(Neu5Ac α2-6LacNAcβ-p-aminophenyl/γ-PGA))

(1) Synthesis of LN-pNP (p-nitrophenyl-LacNAc)

After 75 ml of a solution which included 100 mM tris-HCl (pH 8.0), 20 mM MgCl₂, 20 mM GlcNAc-pNP, 30 mM UDP-Gal, 5.0% (v/v) Acetonitrile, and 0.1 U/ml β1,4-Ga1T, was incubated for 6 hours at 37 degrees C., it was boiled for 5 minutes, divided using centrifugal separation (8000 rpm, 20 minutes) and supernatants were collected. The solution was applied on an ODS column (60 mL, equilibrated by 50 mM triethylamine hydrogencarbonate), and the desired substance was eluted with 5 to 10% MeOH-50 mM triethylamine hydrogencarbonate. LN-pNP containing fractions were collected and after the collected fractions were concentrated, they were azeotropically boiled with water five times and the triethylamine hydrogencarbonate was removed. 307 mg of LN-αPGA was then obtained by drying in a vacuum (20 degrees C., 3 hours).

(NMR of the Obtained LN-pNP)

¹H-NMR (D₂O): δ 8.25 (2H, d, J=9.3 Hz), 7.20 (2H, d, J=9.3 Hz)), 5.36 (1H, d, J=8.4 Hz), 4.53 (1H, d, J=7.8 Hz), 4.12-3.57 (12H, m), 2.03 (3H, s)

### (2) Synthesis of LN-pAP (p-aminophenyl-LacNAc)

LacNAc-pNP (550 mg, 1.09 m mol) was dissolved in a water-methanol mixture (10:1, 44 mL) and 10% carbon supported palladium catalyst (55 mg) and ammonium formate (550 mg, 8.7 m mol) were added and stirred at room temperature for 1.5 hours. A reactive suspended solution was filtrated and the filtrate was concentrated. The residue was applied on an ADS column (80 mL) and the desired substance was eluted with 5% methanol. The solvent was distilled away and 513 mg (99%) of LN-pAP was obtained.

(NMR of the Obtained LN-pAP)

¹H-NMR (D₂O): δ 6.94 (2H, d, J=8.8 Hz), 6.81 (2H, d, J=8.8 Hz), 5.02 (1H, d, J=8.5 Hz), 4.51 (1H, d, J=7.8 Hz), 4.02-3.54 (12H, m), 2.05 (3H, s)

### (3) Synthesis of LN-γ PGA (Poly(LacNAcβ-p-aminophenyl/γ-PGA))

γ-PGA (6.5 mg, 0.043 m mol as glu unit) was dissolved in 100 mM Na₂CO₃/NaHCO₃ buffer, pH 10.0 (0.5 ml). 100 mM Na₂CO₃/NaHCO₃ buffer (0.4 ml) of LN-pAP (60.0 mg, 0.126 m mol), and DMSO solution (1.4 ml) of HOBt (6.5 mg, 0.042 m mol) and BOP reagent (50.7 mg, 0.115 m mol) were each added and the reaction solution was stirred at room temperature for 24 hours and made to react. 2.3 ml of PBS (10 mM phosphate buffer (pH 7.5) and 120 mM NaCl, 2.7 mM KCl) was added and stirred for 2 hours on ice. The sedimentation that arose was eliminated by centrifugal separation (15000 rpm, 5 minutes). 1.5 ml of PSB was added to 4.6 ml of supernatants, and after confirmation that no sedimentation had arisen, the reaction solution was concentrated to 0.8 ml by an evaporator condenser (bath temperature 40 degrees C.) and applied on a gel filtration (Sephadex G—50 F, 8 ml). After a sample was applied, the solution was eluted with 10 ml of ultrapure water, and the total volume of applied sample was collected. The collected sample was put into a dialysis tube and dialyzed against 1000 ml of distilled water and ultrapure water. The dialyzed sample was collected and concentrated to 0.8 ml by an evaporator condenser (bath temperature 40 degrees C.), and 19.0 mg of LN-γ PGA was obtained by lyophilization (shelf temperature 20 degrees C., one night). ¹H-NMR analysis was performed on the obtained LN-γ PGA and the sugar residue substitution rate was calculated as 50% based on the formula below (see FIG. 14)

Sugar residue substitution rate (%)=(A×100)/(B−(3A/4))

(NMR of the Obtained LN-γ PGA)

¹H-NMR (D₂O 60 degrees C.): δ 7.30 (brs), 6.97 (brs), 5.05 (brs), 4.50-3.64 (m), 2.84 (br), 2.43-1.92 (m)

### (4) Synthesis of 3′-SLN-γ PGA(Poly(Neu5Ac α2-3LacNAcβ-p-aminophenyl/γ-PGA))

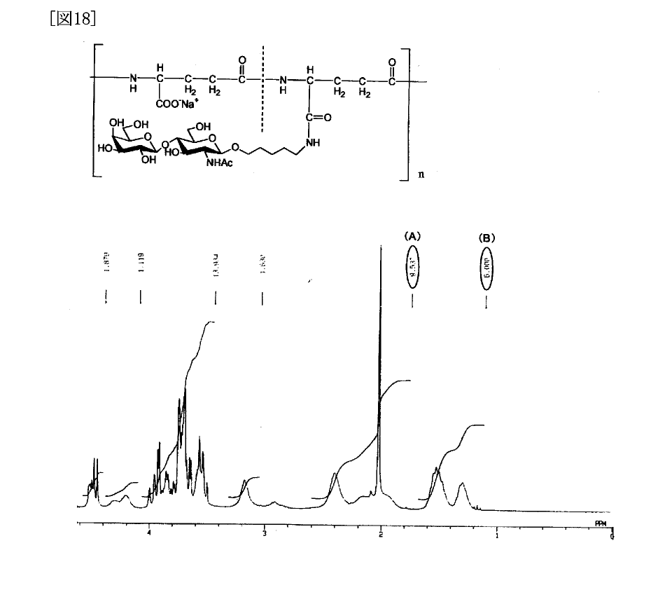

After 1.05 ml of a solution which included 50 mM cacodylic acid buffer (pH 6.0), 2.5 m of MnCl₂, 8 mg of LAcNAc-γ-PGA, 30 mM CMP-NeuAc, 0.1% (w/v) BSA, and 20 U/ml AP, 0.02 U/ml α2-3-SiaT (Rat, Recombinant, Spodoptera frugiperda, CALBICHEM) was incubated for 44 hours at 37 degrees C., it was then boiled for 3 minutes, divided using centrifugal separation (15000 rpm, 5 minutes) and supernatants were collected. The supernatants were applied on a gel filtration (Sephadex G—50 F, 8 ml). After a sample was applied, the solution was eluted with 8 ml of ultrapure water, and the total volume of applied sample was collected. The collected sample was put into a dialysis tube and dialyzed against 1000 ml of distilled water and ultrapure water. The dialyzed sample was collected and applied on an ion exchange column (Dowe×AG 50 W-8×, 3 ml). The sample was then eluted with 30 ml of ultrapure water after being stuck and the total volume was collected from the stuck solution (45 ml). The collected solution was concentrated to 0.8 ml by an evaporator condenser (bath temperature 40 degrees C.) and 9.0 mg of 3′-SLN-γ PGA was obtained by lyophilization (shelf temperature 20 degrees C., one night). The obtained 3′-SLN-γ PGA was analyzed by ¹H-NMR and the sugar residue substitution rate was calculated as 99% based on the formula below (see FIG. 15).

Sialylation rate (%)=(B×100)/(A/4)

(NMR of the obtained 3′-SLN-γ PGA)

¹H-NMR (D₂O 60 degrees C.): δ 7.35 (brs), 7.03 (brs), 5.12 (brs), 4.58 (d, J=7.6 Hz), 4.13-3.54 (m), 2.77 (d, J=12.0 Hz), 2.53-1.91 (m), 1.80 (t, J=12.1 Hz)

### (5) Synthesis of 6′-SLN-γ PGA(Poly(Neu5Ac α2-6LacNAcβ-5-aminophenyl/γ-PGA))

After 1.05 ml of a solution which included 50 mM cacodylic acid buffer (pH6.0), 2.5 m MnCl₂, 8 mg of LAcNAc-γ-PGA, 30 mM CMP-NeuAc, 0.1% (w/v) BSA, 20 U/ml AP, 0.02 U/ml α2-6-SiaT (Rat, Recombinant, Spodoptera frugiperda, CALBICHEM) was incubated for 44 hours at 37 degrees C., it was then boiled for 3 minutes, divided using centrifugal separation (15000 rpm, 5 minutes) and supernatants were collected. The supernatants were applied on a gel filtration (Sephadex G—50 F, 8 ml). After a sample was applied, the solution was eluted with 8 ml of ultrapure water, and the total volume of applied sample was collected. The collected sample was put into a dialysis tube and dialyzed against 1000 ml of distilled water and ultrapure water. The dialyzed sample was collected and applied on an ion exchange column (Dowe×AG 50 W-8×, 3 ml). The sample was then eluted with 30 ml of ultrapure water after being stuck and the total volume was collected from the stuck solution (45 ml). The collected solution was concentrated to 0.8 ml by an evaporator condenser (bath temperature 40 degrees C.) and 7.4 mg of 6′-SLN-γ PGA was obtained by lyophilization (shelf temperature 20 degrees C., one night). The obtained 6′-SLN-γ PGA was analyzed by ¹H-NMR and the sugar residue substitution rate was calculated as 99% based on the formula below (see FIG. 16).

Sialylation rate (%)=(B×100)/(A/4)

(NMR of the obtained 6′-SLN-γ PGA)

¹H-NMR (D₂O 60 degrees C.): δ 7.36 (brs), 7.05 (brs), 5.14 (brs), 4.49-4.39 (m), 4.16 (brs), 4.01-3.55 (m), 2.71 (d, J=9.9 Hz), 2.59-1.81 (m), 1.71 (t, J=12.1 Hz)

### Example 3

### (1) Enzyme

A cellulase (XL-522) originating from Trichoderma resei was purchased from Nagase Chemtex Corporation. α2-3-(N)-sialyltransferase (Rat, Recombinant, Spodoptera frugiperda) and α2-6-(N)-sialyitransferase (Rat, Recombinant, Spodoptera frugiperda) were purchased from CALBIOCHEM. Alkaliphosphatase was purchased from Boehringer Mannheim.

### (2) Substrate

Lactose Monohydrate and 5-amino-1-pentanol were purchased from Wako Pure Chemical Industries. γ-PGA, CMP-Neu5Ac and LacNAc were used by purifying commercially available products according to necessity.

### (3) Reagent

Trifluoroacetic Anhydride and MnCl₂ 4H₂O were purchased from Wako Pure Chemical Industries. BOP, HOBt and BSA were purchased from Sigma—Aldrich.

### (4) Enzyme Activity Assay Method

<Hydrolysis Activity of Lac β-pNP>

In an enzyme activity assay method of cellulase from T. reesei, the amount of released pNP from Lacβ-pNP was determined. 10 mM Lacβ-pNP (25 μl) and 50 mM sodium acetate buffer pH 5.0 (70 μl) were mixed and an appropriate amount of enzymes were added making the total amount 100 μl and made to react at 40 degrees C. for 20 minutes. 10 μl was taken from the reaction solution over time and mixed with 1.0 M sodium carbonate solution (190 μl) which was dispensed in advance in each of the wells of a 96 well micro-plate, and after stopping the reaction, the absorbency at 405 nm was soon determined using a plate reader and the amount of released pNP was determined. The enzyme activity 1 U was defined as the amount of enzymes which release 1 μl mol of pNP in 1 minute.

<Hydrolysis Activity of Gal β-pNP>

In an activity assay method of β-D-galactosidase contained within the cellulase from T. reesei, the amount of released pNP from Galβ-pNP is determined. 10 mM Galβ-pNP (25 μl) and 50 mM sodium acetate buffer pH 5.0 (70 μl) were mixed and an appropriate amount of enzymes were added making the total amount 100 μl and made to react at 40 degrees C. for 20 minutes. 10 μl was taken from the reaction solution over time and mixed with 1.0 M sodium carbonate sodium (190 μl) which was dispensed in advance in each of the wells of a 96 well micro-plate, and after stopping the reaction, the absorbency at 405 nm was soon determined using the plate reader and the amount of released pNP was determined. The enzyme activity 1 U was defined as the amount of enzymes which release 1 μl mol of pNP in 1 minute.

### (5) Enzyme Preparation

<Partial Purification of Cellulase Originating from T. reesei>

After treating a crude enzyme solution (1000 ml, 875 kU) of cellulase originating from T. reesei with 25% saturated ammonium sulphate, centrifugal separation was performed at 4 degrees C. using a high speed micro centrifuge (KUBOTA 1720; RA-200j using a rotor, made by KUBOTA), and supernatants were collected. This was then treated with 75% saturated ammonium sulphate, centrifugal separation was performed at the same conditions and the sedimentation that was produced was dissolved in 10 mM of a sodium phosphate buffer (pH 6.0). After demineralization using an ultrafiltration membrane (PM-30, Millipore Corp) with a molecular weight cut off of 30000, lyophilization was performed and 7.8 g of an enzyme powder was obtained. From this 1.0 g was dissolved in 10 mM of a sodium phosphate buffer (pH 6.0) and brought to a DEAE—Sepharose Fast Flow column chromatography (ø2.6×18 cm) with a column equilibrated in advance with the same buffer. After washing the column with 1000 ml of the same buffer, stepwise elution was performed with 600 ml of the same buffer containing 500 mM NaCl. After demineralization of the column absorbed fraction by ultrafiltration and concentrating, lyophilization was performed and a partial purified enzyme (0.7 g, 0.70 U/mg) was obtained.

### <Removal of β-D Galactosidase by Using Gal-Amidine Gel>

The partial purified enzyme (50 mg, Lac β-pNP hydrolysis activity 35 U, Gal β-pNP hydrolysis activity 19 U) was dissolved in 50 mM sodium phosphate buffer pH 6.0 (1.0 ml) and brought to a Gal-amidine affinity column chromatography (ø1.2×1.7 cm) with a column equilibrated in advance with the same buffer. At a flow rate of 10 ml/h, 1 ml was put into each Eppendorf tube and the non-absorbed fractions were washed off with the 50 mM sodium phosphate buffer pH 6.0 (30 ml). The absorbed fraction was eluted with 50 mM sodium phosphate buffer pH 6.0 (20 ml) which included 1.0 M NaCl, and was further eluted with 50 mM sodium acetate buffer pH 4.0 (10 ml) which included 0.5 M methyl β-Gal. Detection of proteins was carried out by assaying the absorbency at 280 nm and the hydrolysis activity of Lac β-pNP and Gal β-pNP was assayed. After each fraction was concentrated using an ultrafiltration membrane (PM-30, Millipore Corp) with a molecular weight cut off of 30000, lyophilization was performed and a partial purified enzyme (Lac β3-pNP hydrolysis activity 32 U, Gal β-pNP hydrolysis activity 0.3 U) in which β-D galactosidase was removed from the non absorbed fraction, was obtained (Table. 1). Furthermore, all partial purified enzymes in which β-D galactosidase was removed was used in further reactions.

Poly (Neu5Aca α2-3LacNAc β-5-aminopentyl/γ-PGA) and Poly (Neu5Aca α2-6LacNAc β-5-aminopentyl/γ-PGA) were prepared in the order cited in the synthesis path shown in the following formula (X) using these enzymes and the like.

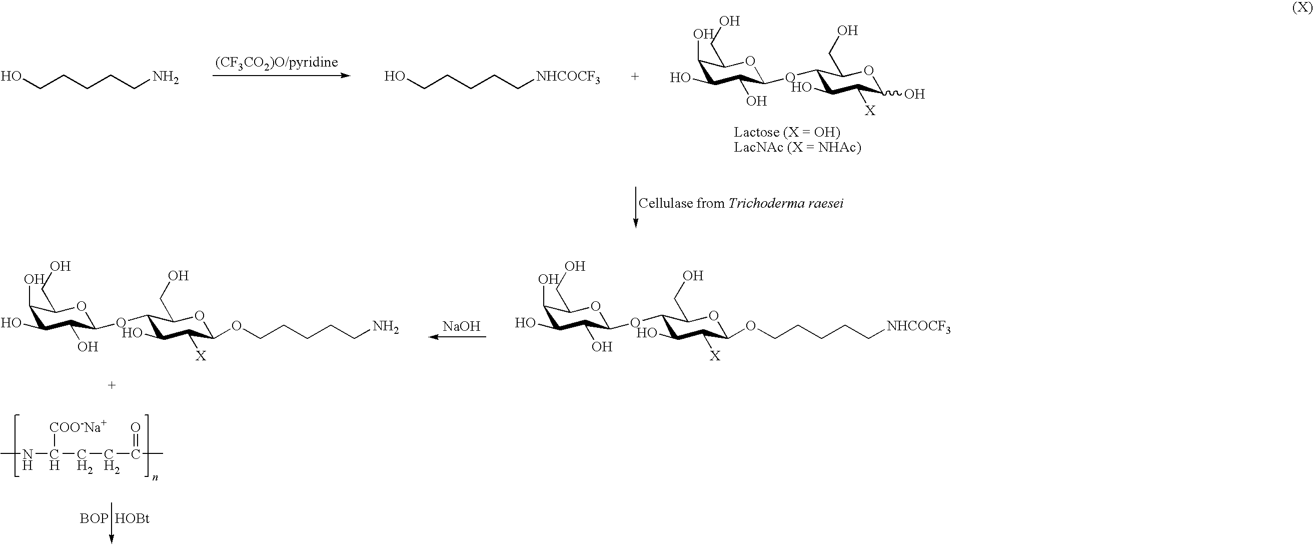

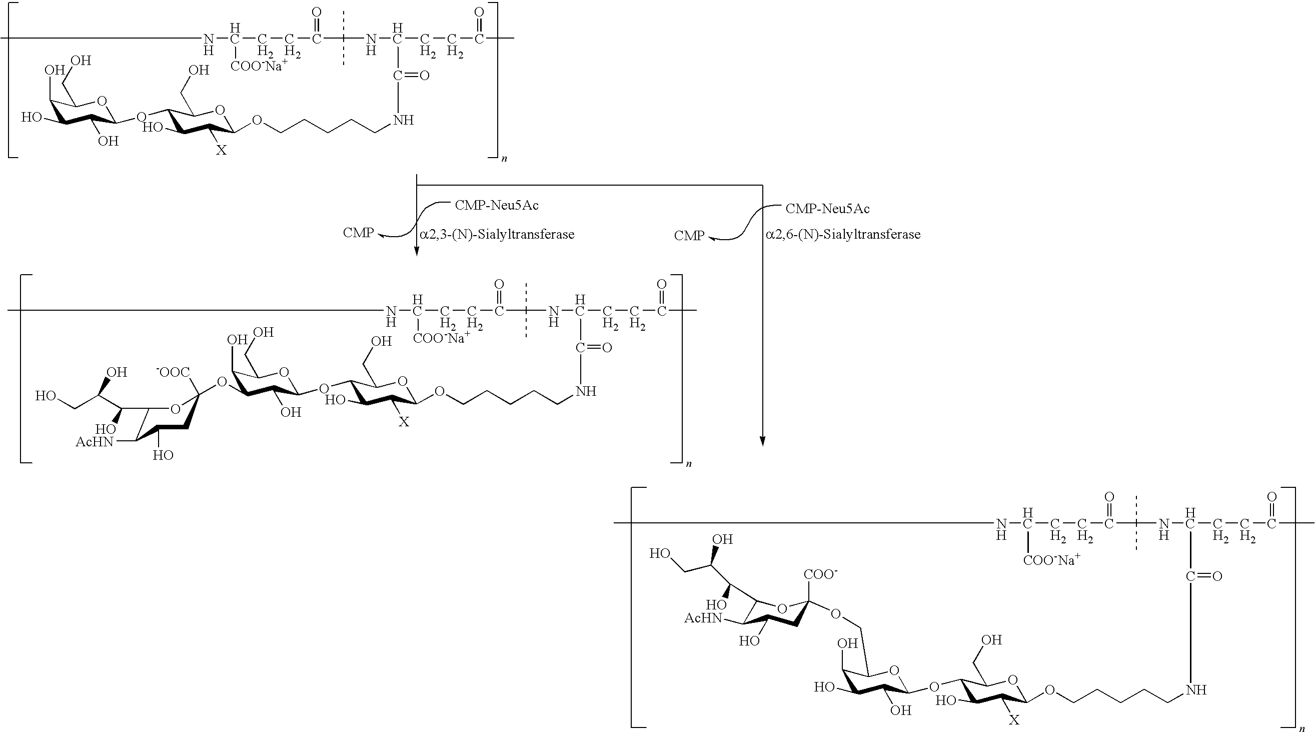

### (6) Chemical Synthesis of 5-Trifluoroacetamido-1-pentanol

At first, pyridine (20 ml) was added to 5-amino-1-pentanol (10 g, 97 m mol) and dissolved. This was then cooled on ice and stirred and anhydrous trifluoroacetic acid (25 ml, 180 m mol) was attached in drops and allowed to begin to react. Every 5 minutes from the start of the reaction the reaction was confirmed using phosphomolybdic acid color reaction by TLC (developing solvent; chloroform: acetone=8:2). Following confirmation that the raw material had disappeared after one hour, crushed ice of about the same amount as the reaction solution was added to stop the reaction, and then, 20 ml of saturated sodium hydrogencarbonate aqueous solution was added and the reaction solution was neutralized. After concentrating the reaction solution, an appropriate amount of acetone was added and again concentrated. After repeating this operation about three times, the reaction solution was dissolved in acetone and a large quantity of sodium hydrogen carbonate which existed was separated out. After filtering these, they were concentrated and treated with silica-gel chromatography (ø4.5×35 cm) which was equilibrated (10 ml/min) by chloroform: acetone=8:2. Mobile phases which passed through the column were sampled at about every 25 ml. The eluted fractions were checked to confirm substances produced using phosphomolybdic acid color reaction by TLC (developing solvent; chloroform: acetone=8:2). The fraction which contained the desired substance was concentrated and 18 g of the desired 5-Trifluoroacetamido-1-pentanol was obtained with a 94% yield.

¹H-NMR was then performed.

(NMR of 5-Trifluoroacetamido-1-pentanol)

¹H-NMR (D₂O, 270 MHz): δ 3.59 (t, 2H, H-α), 3.33 (t, 2H, H-e), 1.65-1.48 (2H×2, H-b, d), 1.36 (2H, H-c)

### (7) Synthesis of 5-Trifluoroacetamidopentyl β-lactoside

Lactose (54.3 g, 151 m mol) and Trifluoroacetamido-1-pentanol (30.0 g, 151 m mol) as substrates were dissolved in 50 mM sodium acetate buffer pH 5.0 (151 ml), cellulase (4500 U) originating from T. reesei in which galactosidase was removed was added and made to react. In order to keep track of the reaction, 10 μl of the reaction solution was collected over a period of time and after 190 μl of demineralized water was added, the solution was boiled for 10 minutes at 100 degrees C. to stop the reaction, and after filtering with a 0.45 μm filter the filtered solution was analyzed by HPLC. The reaction solution was shaken intensively (200 rpm) and made to react for 120 hours at 40 degrees C. Following this, the reaction was stopped by boiling for 10 minutes at 100 degrees C. After concentrating the reaction solution, the concentrated solution was applied on a silica-gel 60N column chromatography (ø4.5×50 cm) process in which a column was equilibrated by a solvent (10 ml/min) of chloroform:methanol: water=7:3:0.5, and was eluted with the same solvent, separated to take 23 ml into each tube and analyzed by TLC (chloroform:methanol: water=7:3:0.5). The fraction which contained the desired fractions was concentrated, dissolved in heavy water and analyzed by ¹H-NMR to find that 849 mg of 5-Trifluoroacetamidopentyl β-lactoside was obtained with a 1.0% yield.

### (NMR of 5-Trifluoroacetamidopentyl β-Lactoside)

¹H-NMR (D₂O, 270 MHz): δ 4.48 (d, 1H, H-1), 4.45 (d, 1H, H-1), 3.34 (t, 2H, H-e), 3.32 (1H, H-2), 1.71-1.57 (2H×2, H-b, d), 1.42 (2H, H-c)

### (8) Synthesis of 5-Trifluoroacetamidopentyl β-N-acetyllactosaminide

N-acetyllactosaminide (20.0 g, 52.2 m mol) and 5-Trifluoroacetamido-1-pentanol (15.6 g, 78.4 m mol) as substrates were dissolved in 100 mM sodium acetate buffer pH 4.0 (52.2 ml), cellulase (6200 U) originating from T. reesei in which galactosidase was removed was added and made to react. In order to trace the reaction 10 μl of the reaction solution was collected over a period of time and after 190 μl of demineralized water was added, the solution was boiled for 10 minutes at 100 degrees C. to stop the reaction, and after filtering with a 0.45 μm filter the filtered solution was analyzed by HPLC. The reaction solution was shaken intensively (200 rpm) and made to react for 144 hours at 40 degrees C. Following this, the reaction was stopped by boiling for 10 minutes at 100 degrees C. After concentrating the reaction solution, active carbon-sellite chromatography (ø4.5×100 cm) with a column which was equilibrated (5.0 ml/min) with water was performed. First, LacNAc which was used as the substrate was eluted with linear gradient method of ethanol 0% (5.0 L) to 25% (5.0 L). After taking 60 ml into each tube, each fraction was assayed at the absorbency of 210 nm which originates from an N-acetyl group. A recovered amount of LacNAc was 17.2 g and a recovered yield was 86% by concentrating the fraction which contained LacNAc. Next, an absorbed fraction was eluted with switching to 80% ethanol (5.0 L). After taking 60 ml into each tube, each fraction was assayed at the absorbency of 210 nm. Then, the fraction which contained the desired fraction was concentrated, the concentrated solution was treated with silica-gel 60N column chromatography (ø4.5×50 cm) which was equilibrated with a solvent (10 ml/min) of chloroform:methanol:water=7:3:0.5, and was eluted with the same solvent, separated to take 28 ml into each tube and analyzed by TLC (chloroform:methanol: water=7:3:0.5). The fraction which contained the desired fraction was concentrated, dissolved in heavy water and analyzed by ¹H-NMR to find that 322 mg of 5-Trifluoroacetamidopentyl β-N-acetyllactosaminide was obtained with a 1.1% yield.

### (NMR of 5-Trifluoroacetamidopentyl β-N-acetyllatosaminide)

¹H-NMR (D₂O, 270 MHz): δ 4.51 (d, 1H, H-1), 4.46 (d, 1H, H-1), 3.31 (t, 2H, H-e), 2.02 (s, 3H, —NHAc), 1.57 (2H×2, H-b, d), 1.34 (2H, H-c)

### (9) Synthesis of 5-aminopentyl β-lactoside

1.0 M NaOH (1.2 ml) was added to 5-Trifluoroacetamidopentyl β-lactoside (104 mg, 0.19 m mol) and dissolved and a reaction was started at room temperature. The reaction was confirmed using orcinol sulfate color reaction and phosphomolybdic acid color reaction by TLC (developing solvent; chloroform:methanol:water=7:3:0.5) every 30 minutes from the start of the reaction. After it was confirmed by TLC that the raw material had dissapeared 1 hour after the start, a Sephadex G-25 column chromatography (ø4.5×50 cm) with a column which was equilibrated with water (1.0 ml/min) was performed. Mobile phases which passed through the column about every 2.0 ml were sampled. The eluted fractions were checked to confirm substances produced using phosphomolybdic acid color reaction by TLC (developing solvent; chloroform:methanol:water=7:3:0.5). The fraction which contained the desired substance was concentrated and 18 g of the desired 5-Trifluoroacetamido-1-pentanol was obtained with a 94% yield. ¹H-NMR was then performed.

(NMR of 5-aminopentyl β-lactoside)

¹H-NMR (D₂O, 500 MHz): δ 4.49 (d, 1H, H-1), 4.45 (d, 1H, H-1), 3.30 (t, 1H, H-2), 2.97 (t, 2H, H-e), 1.67 (2H×2, H-b, d), 1.47 (2H, H-c)

### (10) Synthesis of 5-aminopentyl β-N-acetyllactosaminide

1.0 M NaOH (1.2 ml) was added to 5-Trifluoroacetamidopentyl β-N-acetyllactosaminide (100 mg, 0.18 m mol) and dissolved and a reaction was started at room temperature. The reaction was confirmed using orcinol sulfate color reaction and phosphomolybdic acid color reaction by TLC (developing solvent; chloroform:methanol:water=6:4:1) every 30 minutes from the start of the reaction. After it was confirmed by TLC that the raw material had disappeared 1 hour after the start, Sephadex G-25 column chromatography (ø2.5×55 cm) with a column which was equilibrated with water (1.0 ml/min) was performed. Mobile phases which passed through the column about every 2.0 ml were sampled. The eluted fractions were checked to confirm substances produced by the absorbency of 210 nm which originates in an N-acetyl group and phosphomolybdic acid color reaction by TLC (developing solvent; chloroform:methanol:water=6:4:1). The fraction which contained the desired substance was concentrated and 82 g of the desired 5-aminopentyl β-N-acetyllactosaminide was obtained with a 99% yield. ¹H-NMR was then performed.

(NMR of 5-aminopentyl β-N-acetyllactosaminide)

¹H-NMR (D₂O, 270 MHz): δ 4.52 (d, 1H, H-1), 4.47 (d, 1H, H-1), 2.77 (t, 2H, H-e), 2.03 (s, 3H, —NHAc), 1.54 (2H×2, H-b, d), 1.35 (2H, H-c)

### (11) Synthesis of Poly (5-aminopentyl β-lactoside/γ-PGA)

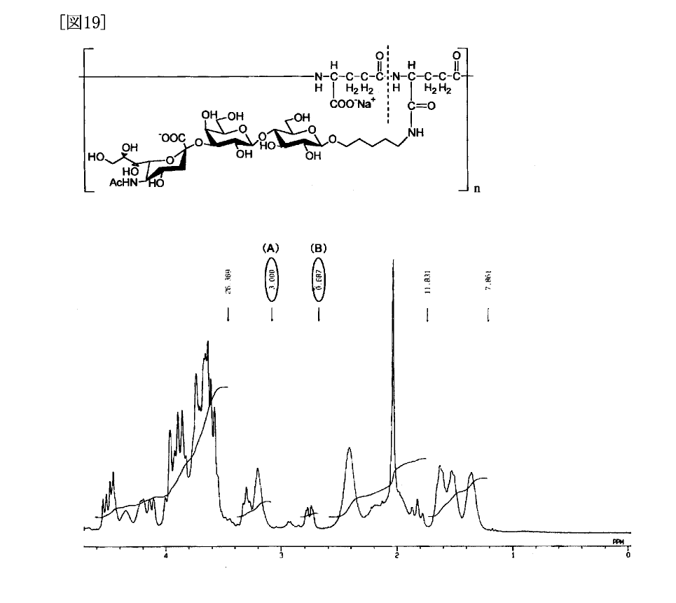

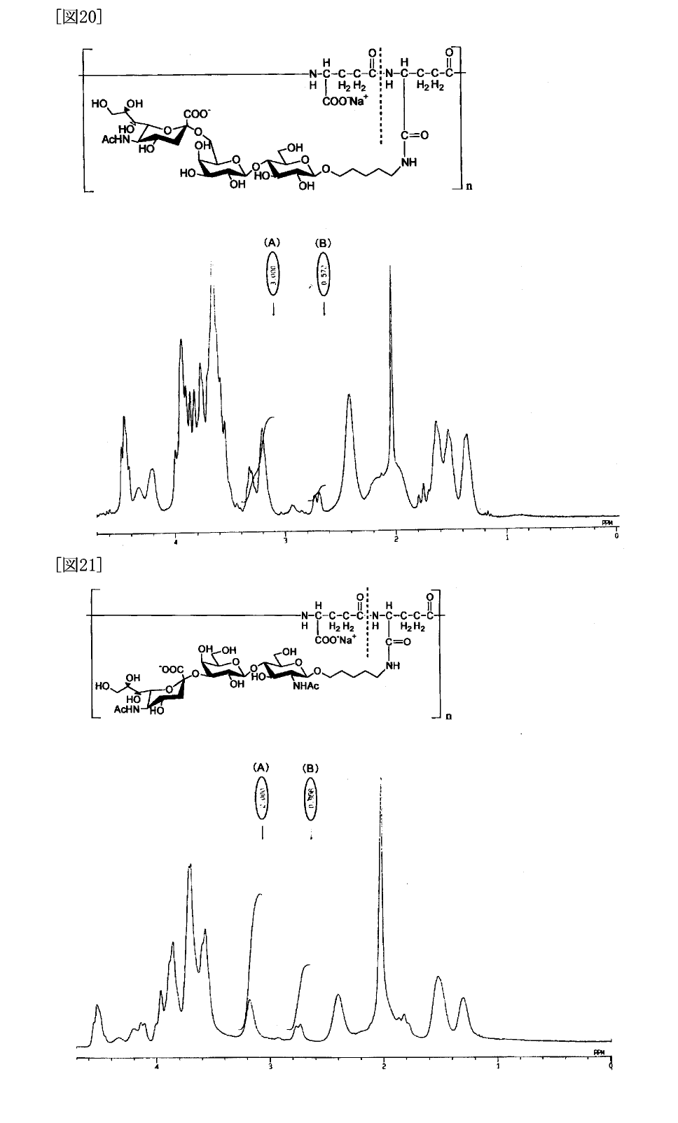

After γ-PGA (M.W.: 77000, 16.5 mg) was dissolved in 100 mM Na₂CO₃/NaHCO₃ pH 10.0 (1.3 ml), BOP (130 mg) and HOBt (16 mg) which had been dissolved in advance in DMSO (3.5 ml) were added and stirred using a stirrer. Lastly, after 5-aminopentyl β-lactoside (140 mg) was dissolved in Na₂CO₃/NaHCO₃ pH 10.0 (0.9 ml), a reaction was done for 24 hours at room temperature while dropping and stirring. After the reaction was completed, PBS was added so that the reaction solution became 7.5 ml. After this, 2.5 ml of the reaction solution per PD-10 column was applied on a PD-10 column (ø0.7×5.0 cm, Sephadex G-25) which had equilibrated with PBS and Poly (5-aminopentyl β-lactoside/γ-PGA) was eluted with 3.5 ml of PBS. Next, this fraction was dialyzed for 3 days against 2.5 L of ultrapurified water. During that time, the ultrapurified water was changed six times. In addition, after the dialysis, the sample was concentrated and lyophilized. Next, a structural analysis was performed by ¹H-NMR. In addition, the sugar residue substitution rate (%) was calculated by applying an integration rate (A) of protons of β and γ positions of γ-PGA and an integration rate (B) of 6 protons of the agylcon position of 5-aminopentyl β-lactoside to the formula shown below (FIG. 17) using the ¹H-NMR results. As a result, it was found that 29.6 mg of Poly (5-aminopentyl β-lactoside/γ-PGA) with a 69% sugar residue substitution rate was obtained.

Sugar residue substitution rate (%)=(4×100)/(A−(B/6))

(NMR of Poly (5-aminopentyl β-lactoside/γ-PGA))

¹H-NMR (D₂O, 500 MHz): δ 4.47 (d, 1H, H-1), 4.45 (d, 1H, H-1), 4.34-4.22 (1H, H-α), 3.31 (t, 1H, H-2), 3.20 (2H, H-e), 2.42 (2H, H-γ), 2.20-1.98 (2H, H-β), 1.63 (2H, H-d), 1.52 (2H, H-b), 1.35 (2H, H-c)

### (12) Synthesis of Poly (5-aminopentyl β-acetyllactosaminide/γ-PGA)

After γ-PGA (M. W.: 77000, 15.1 mg) was dissolved in 100 mM Na₂CO₃/NaHCO₃ pH 10.0 (1.3 ml), BOP (119 mg) and HOBt (15 mg) which had been dissolved in advance in DMSO (3.5 ml) were added and stirred using a stirrer. Lastly, after 5-aminopentyl β-acetyllactosaminide (140 mg) was dissolved in Na₂CO₃/NaHCO₃ pH 10.0 (0.9 ml), a reaction was done for 24 hours at room temperature while dropping and stirring. After the reaction was completed, PBS was added so that the reaction solution became 7.5 ml. After this, 2.5 ml of the reaction solution per PD-10 column was applied on a PD-10 column (ø1.7×5.0 cm, Sephadex G-25) which had equilibrated with PBS and Poly (5-aminopentyl β-acetyllactosaminide/γ-PGA) was eluted with 3.5 ml of PBS. Next, this fraction was dialyzed for 3 days against 2.5 L of ultrapurified water. During that time, the ultrapurified water was changed six times. After the dialysis, the sample was concentrated and lyophilized. Next, a structural analysis was performed by ¹H-NMR. In addition, the sugar residue substitution rate (%) was calculated by applying an integration rate (A) of protons of β and γ positions of γ-PGA and an integration rate (B) of 6 protons of the agylcon position of 5-aminopentyl β-acetyllactosaminide to the formula shown below (FIG. 18) using the ¹H-NMR results. As a result, it was found that 17.0 mg of Poly (5-aminopentyl β-acetyllactosaminide/γ-PGA) with a 61% sugar residue substitution rate was obtained.

In addition, as a result of using the same composition as that stated above and γ-PGA (M.W.: 990000, 15.0 mg) in order to synthesize a higher molecular weight sugar chain polypeptide with aisalo disaccharide, 24.0 mg of Poly (5-aminopentyl β-acetyllactosaminide/γ-PGA) with a 58% sugar residue substitution rate was obtained. The sugar residue substitution rate was calculated as in the following formula.

Sugar residue substitution rate (%)=(4×100)/(A−(B×6))

(NMR of Poly (5-aminopentyl β-lactoside/γ-PGA))

¹H-NMR (D₂O, 270 MHz): δ 4.51 (d, 1H, H-1), 4.47 (d, 1H, H-1′), 4.30-4.21 (1H, H-α), 3.18 (2H, H-e), 2.40 (2H, H-γ), 2.18-1.98 (2H, H-β), 2.02 (s, 3H, —NHAc), 1.52 (2H×2, H-b, d), 1.35 (2H, H-c)

### (13) Synthesis of Poly (Neu5Ac α2-3Lac β-5-aminopentyl/γ-PGA)

5.5 mg of Poly (5-aminopentyl β-lactoside/γ-PGA) [69%, 210 kDa] as an acceptor substrate was prepared so that the preparation became 8.0 mM per one Lac unit and, 16.0 mM CMP-Neu5Ac as a donor substrate, 2.5 mM MnCl₂, 0.1% BSA, and 50 mM MOPS buffer (pH7.4) were prepared. Next, 10 U/ml of alkaline phosphatase and 40 mU/ml of α2-3-(N)-sialyltransferase were added to a reaction solution and a reaction was allowed to occur for 48 hours at 37 degrees C. The rate of sialylation was calculated by applying the sum (A) of an integration rate of Glc (H-2) proton originating in a sugar chain and an integration rate of 2 protons of the agylcon position of 5-aminopentyl β-acetyllactosaminide, and an integration rate (B) of proton of the third equatorial position which is characteristic of Neu5Ac to the formula below using the ¹H-NMR results. As a result, it was found that 6.7 mg of Poly (Neu5Ac α2-3Lac β-5-aminopentyl/γ-PGA) with a 69% rate of sialylation was obtained.

Rate of sialylation (%)=(B×100)/(A/3)

(NMR of Poly (Neu5Ac α2-3Lac β-5-aminopentyl/γ-PGA))

¹H-NMR (D₂O, 270 MHz): δ 4.53 (d, 1H, H-1), 4.47 (d, 1H, H-1′), 4.35-4.19 (1H, H-α), 3.30 (t, 1H, H-2), 3.20 (2H, H-e), 2.76 (dd, 1H, h-3″ eq), 2.41 (2H, H-γ), 2.20-1.98 (2H, H-β), 2.03 (s, 3H, —NHAc″), 1.82 (t, 1H, H-3″ ax), 1.63 (2H, H-d), 1.53 (2H, H-b), 1.36 (2H, H-c)

### (14) Synthesis of Poly (Neu5Ac α2-6Lac β-5-aminopentyl/γ-PGA)

5.5 mg of Poly (5-aminopentyl β-lactoside/γ-PGA) [69%, 210 kDa] as an acceptor substrate was prepared so that the preparation became 8.0 mM per one Lac unit and, 16.0 mM CMP-Neu5Ac as a donor substrate, 2.5 mM MnCl₂, 0.1% BSA and MOPS buffer (pH7.4) were prepared. Next, 10 U/ml of alkaline phosphatase and 40 mU/ml of α2-6-(N)-sialyltransferase were added to a reaction solution and a reaction was allowed to occur for 48 hours at 37 degrees C. The rate of sialylation was calculated by applying the sum (A) of an integration rate of Glc (H-2) proton originating in a sugar chain and an integration rate of 2 protons of the agylcon position of 5-aminopentyl β-acetyllactosaminide, and an integration rate (B) of proton of the third equatorial position which is characteristic of Neu5Ac to the formula below using the ¹H-NMR results. As a result, it was found that 6.8 mg of Poly (Neu5Ac α2-6Lac β-5-aminopentyl/γ-PGA) with a 57% rate of sialylation was obtained.

Rate of sialylation (%)=(B×100)/(A/3)

(NMR of Poly (Neu5Ac α2-6Lac β-5-aminopentyl/γ-PGA))

¹H-NMR (D₂O, 270 MHz): δ 4.47 (d, 1H, H-1), 4.43 (d, 1H, H-1′), 4.32-4.20 (1H, H-α), 3.32 (t, 1H, H-2), 3.20 (2H, H-e), 2.71 (dd, 1H, h-3″ eq), 2.41 (2H, H-γ), 2.20-1.98 (2H, H-β), 2.03 (s, 3H, —NHAc″), 1.75 (t, 1H, H-3″ ax), 1.63 (2H, H-d), 1.52 (2H, H-b), 1.35 (2H, H-c)

### (15) Synthesis of Poly (Neu5Ac α2-3LacNAc β-5-aminopentyl/γ-PGA)

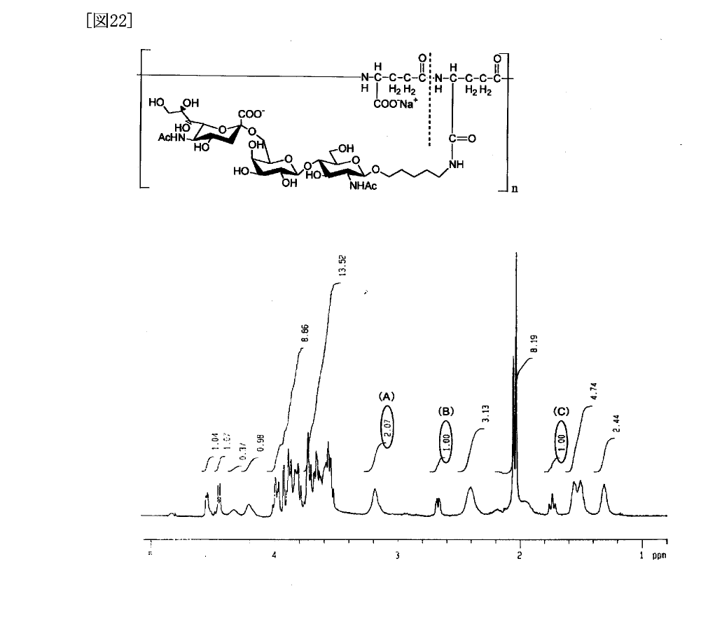

5.0 mg of Poly (5-aminopentyl β-N-acetyllactosaminide/γ-PGA) [61%, 210 kDa] as an acceptor substrate was prepared so that the preparation became 8.0 mM per one Lac unit and, 16.0 mM CMP-Neu5Ac as a donor substrate, 2.5 mM MnCl₂, 0.1% BSA and MOPS buffer (pH 7.4) were prepared. Next, 10 U/ml of alkaline phosphatase and 40 mU/ml of α2-3-(N)-sialyltransferase were added to a reaction solution and a reaction was allowed to occur for 48 hours at 37 degrees C. The rate of sialylation was calculated by applying an integration rate (A) of 2 protons of the agylcon position of 5-aminopentyl β-N-acetyllactosaminide, and an integration rate (B) of proton of the third equatorial position which is characteristic of Neu5Ac to the formula below using the ¹H-NMR results. As a result, it was found that 6.4 mg of Poly (Neu5Ac α2-3LacNAc β-5-aminopentyl/γ-PGA) with a 96% rate of sialylation was obtained (FIG. 21).

In addition, when sialylation was carried out by the same method as that stated above using 5.0 mg of Poly (5-aminopentyl β-N-acetyllactosaminide/γ-PGA) [58%, 2600 kDa] as an acceptor substrate, 6.0 mg of Poly (Neu5Ac α2-3LacNAc β-5-aminopentyl/γ-PGA) with a 100% rate of sialylation was obtained. The rate of sialylation was calculated as in the following formula.

Rate of sialylation (%)=(B×100)/(A−2)

(NMR of Poly (Neu5Ac α2-3LacNAc β-5-aminopentyl/γ-PGA))

¹H-NMR (D₂O, 270 MHz): δ 4.53 (d, 1H, H-1), 4.47 (d, 1H, H-1′), 4.35-4.20 (1H, H-α), 3.18 (2H, H-e), 2.73 (dd, 1H, h-3″ eq), 2.40 (2H, H-γ), 2.20-1.98 (2H, H-β), 2.03 (s, 3H, —NHAc, —NHAc″), 1.82 (t, 1H, H-3″ ax), 1.52 (2H×2, H-b, d), 1.30 (2H, H-c)

(16) Synthesis of Poly (Neu5Ac α2-6LacNAc β-5-aminopentyl/γ-PGA)

5.0 mg of Poly (5-aminopentyl β-N-acetyllactosaminide/γ-PGA) [61%, 210 kDa] as an acceptor substrate was prepared so that the preparation became 8.0 mM per one Lac unit and as a donor substrate, 16.0 mM CMP-Neu5Ac, 2.5 mM MnCl₂, 0.1% BSA and MOPS buffer (pH 7.4) was prepared so that they became the concentrations stated above. Next, 10 U/ml of alkaline phosphatase and 40 mU/ml of α2-6-(N)-sialyltransferase were added to a reaction solution and a reaction was allowed to occur for 48 hours at 37 degrees C. The rate of sialylation was calculated by applying to an integration rate (A) of 2 protons of the agylcon position of 5-aminopentyl β-N-acetyllactosaminide, an integration rate (B) of proton of the third equatorial position which is characteristic of Neu5Ac and an integration rate (C) of proton of the third axial position to the formula below using the ¹H-NMR results. As a result, it was found that 6.1 mg of Poly (Neu5Ac α2-6LacNAc β-5-aminopentyl/γ-PGA) with a 97% rate of sialylation was obtained (FIG. 22).

In addition, when sialylation was carried out by the same method as that stated above using 5.0 mg of Poly (5-aminopentyl β-N-acetyllactosaminide/γ-PGA) [58%, 2600 kDa] as an acceptor substrate, 6.0 mg of Poly (Neu5Ac α2-6LacNAc β-5-aminopentyl/γ-PGA) with a 100% rate of sialylation was obtained. The rate of sialylation was calculated as in the following formula.

Rate of sialylation (%)=((B+C)/2×100)/(A/2)

(NMR of Poly (Neu5Ac α2-6LacNAc β-5-aminopentyl/γ-PGA))

¹H-NMR (D₂O, 500 MHz): δ 4.55 (d, 1H, H-1), 4.45 (d, 1H, H-1′), 4.33-4.21 (1H, H-α), 3.19 (2H, H-e), 2.67 (dd, 1H, h-3″ eq), 2.40 (2H, H-γ), 2.18-1.98 (2H, H-β), 2.06 (s, 3H, —NHAc″), 2.03 (s, 3H, —NHAc), 1.74 (t, 1H, H-3″ ax), 1.52-1.51 (2H×2, H-b, d), 1.31 (2H, H-c)

### Example 4

The two kinds of polymer with sialo-oligosaccharide (sialyl-glycopolymer) stated below which were prepared by a reference example method, were immobilized on a microtiter plate by the following method. First, 100 μl of PBS solution of the polymer with sialo-oligosaccharide was added (multiple dilutions: 200 μg/ml, diluted multiple times with a concentration of PBS as a maximum concentration) to each well of a microtiter plate (Corning—Costar, Labcoat 2503, Cambridge Mass.) having 96 wells. Next, after leaving the plate for one hour at room temperature, the plate was then put onto a glass surface of an ultraviolet ray irradiation apparatus (VILBER LOURMAT, France), and irradiated with ultraviolet rays (254 nm) for one minute. After irradiation, the solution of polymer with sialo-oligosaccharide inside the wells was discarded by tilting the plate. Then, 100 μg of 2% BSA (Sigma, Grade 96%) was added to the plate and a blocking treatment was carried out for one hour at room temperature.

Following this, each well was washed five times with 100 μl of PBS, and 100 μl of a PBS solution containing three kinds of inactivated influenza virus (avian A virus: A/duck/Hong Kong/24/76 (H₃N₂), 32HAU (hemagglutination units); human A virus: A/Memphis/1/71/(H₃N₂), 32HAU; human B virus: B/Lee/40) was added and was left for 12 hours while slowly shaking at 4 degrees C. After washing three times with PBS, 50 μl of an anti influenza virus rabbit antiserum (1000 times diluted) was added to each well and slowly shaken for two hours at 4 degrees C. Then 50 μl of horseradish peroxidase-binding protein A (Organon Teknika N. V Cappel Products, Turnout, Belgium, 1000 times diluted) was added and slowly shaken for two hours at 4 degrees C. After washing each well three times with PBS, 50 μl of a substrate reagent (orthophenylenediamine (Wako Pure Chemicals, Japan) solution including 0.01% H₂O₂) was added, left for ten minutes at room temperature, and next 50 μl of 1N NCl was added and a reaction was stopped. Then, the developed color of each well was colorimetrically determined at 492 nm (control: contrasted with 630 nm).

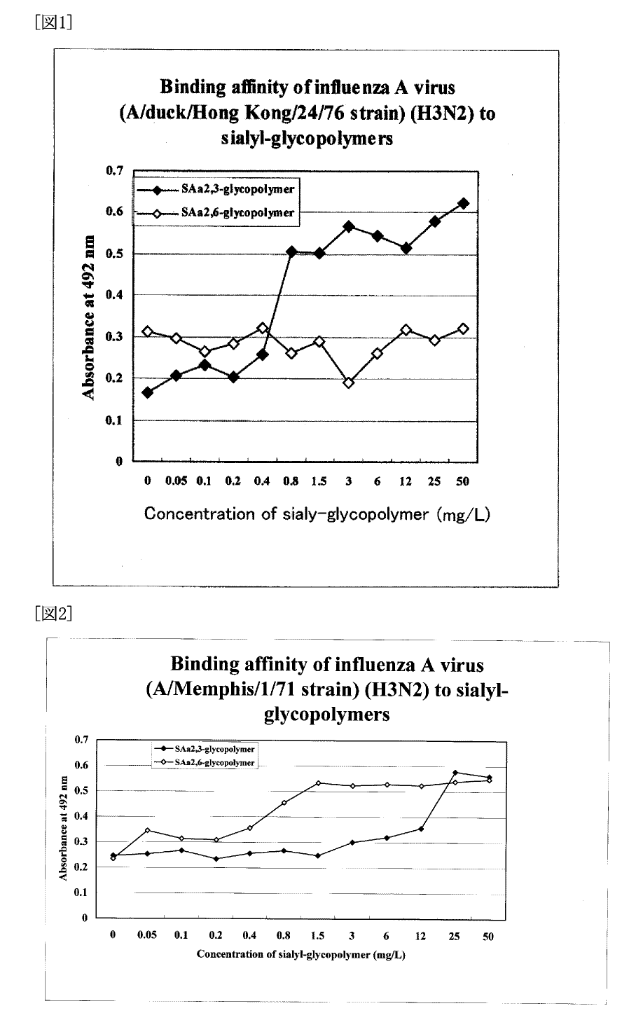

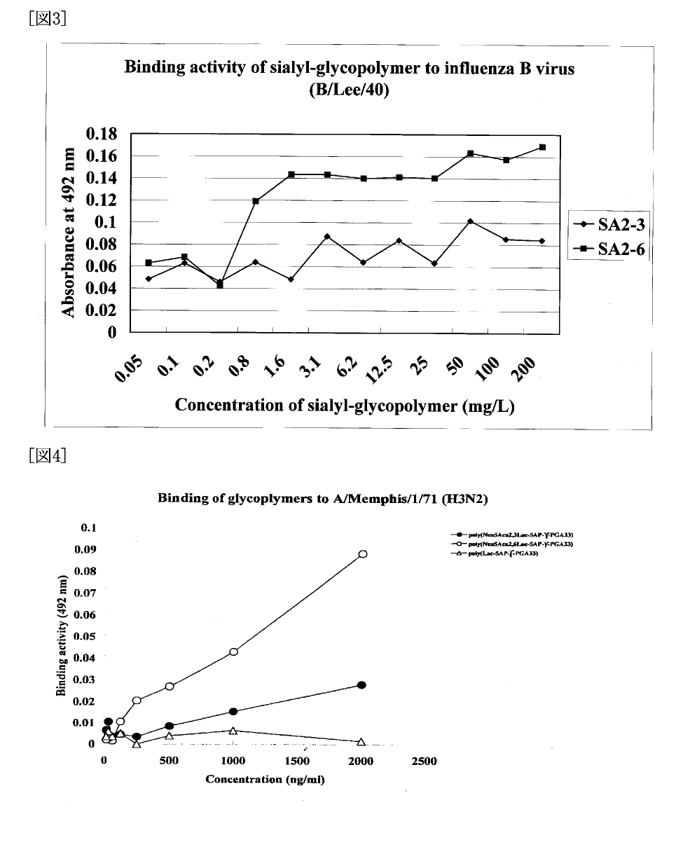

The result of the avian A virus (A/duck/Hong Kong/24/76) (H3N2) is shown in a graph in FIG. 1, the result of the human A virus (A/Memphis/1/71) (H3N2) is shown in a graph in FIG. 2 and the result of the human B virus (B/Lee/40) is shown in a graph in FIG. 3. In FIG. 1 to 3, the vertical axis shows absorbency at 492 nm, and the horizontal axis shows concentration (mg/L) of the polymer with sialo-oligosaccharide. Also, in FIG. 1 to 3, [SAα2,3-glycopolymer] shows a 2-3 type polymer with sialo-oligosaccharide stated below and [SAα2,6-glycopolymer] shows a 2-6 type polymer with sialo-oligosaccharide stated below.

Polymer with Sialo-Oligosaccharide
(2-3 type)

### Poly (Neu5Ac α2-3Gal β1-4GlcNAc β-pAP/α-PGA)

(2-6 type)

### Poly (Neu5Ac α2-6Gal β1-4GlcNAc β-pAP/α-PGA)

As is shown in the graph in FIG. 1, the avian influenza A virus strongly recognizes the 2-3 type of polymer with sialo-oligosaccharide, however, its recognition of the 2-6 type of polymer with sialo-oligosaccharide is weak. In addition, as is shown in the graph in FIG. 2, the human influenza A virus strongly recognizes the 2-6 type of polymer with sialo-oligosaccharide but its recognition of the 2-3 type of polymer with sialo-oligosaccharide is weak. And, as is shown in the graph in FIG. 3, the human influenza B virus strongly recognizes the 2-6 type of polymer with silao sugar chain but its recognition of the 2-3 type of polymer with sialo-oligosaccharide is weak.

### Example 5

After each type of sialo-oligosaccharide binding polyglutamate polymer (2 μg/ml) had been diluted multiple times with a PBS solution, 100 μl was added to each well of a microplate (Corning—Costar; Labcoat 2503, Cambridge, Mass.). Next, after the plate was left to rest for 2 hours at 4 degrees C., the plate was then put onto a glass surface of an ultraviolet ray irradiation apparatus and irradiated with ultraviolet rays (254 nm) for 10 minutes. After irradiation the solution of polymer with sialo-oligosaccharide inside the wells was discarded, 250 μl of 2% BSA solution (Albumin bovine Fraction V, Sigma, St, Louis, Mo.) or 0.01% blockace solution (Dainippon Pharmaceutical) was added to the plate and a blocking treatment was carried out for one night at 4 degrees C. After this, each well was washed five times with 250 μl of PBS and 50 μl of a suspended PBS solution of an influenza virus inactivated by ether treatment (avian A virus: A/duck/Hong Kong/313/4/78 (H5N3), 128 HAU; human A virus: A/Memphis/1/71/(H3N2), 128HAU) was added to each well and left for 5 hours at 4 degrees C. After washing each well with 250 μl of a PBS solution containing 2% Tween20, 50 μl of an anti influenza virus rabbit antiserum which had been diluted 1000 times by 0.1% BSA or 0.01% blockase, was added to each well and left for 2 hours at 4 degrees C. After washing each well with 250 μl of the PBS solution containing 0.01% Tween20, 50 μl of a HRP labeled protein A which had been diluted 1000 times by 0.1% BSA or 0.01% blockase, was added to each well and left for 2 hours at 4 degrees C. After washing each well with 250 μl of the PBS solution containing 0.01% Tween20, 100 μl of a substrate solution (O-phenylenediamine 4 mg, 100 mM phosphate citrate buffer pH P 5.0 including 0.01% H₂O₂) was added and after leaving to rest for 15 to 20 minutes at room temperature, 50 μl of 1N sulphuric acid aqueous solution was added and the reaction was stopped. The developed color of each well was then assayed at 492 nm (control wavelength 630 nm).

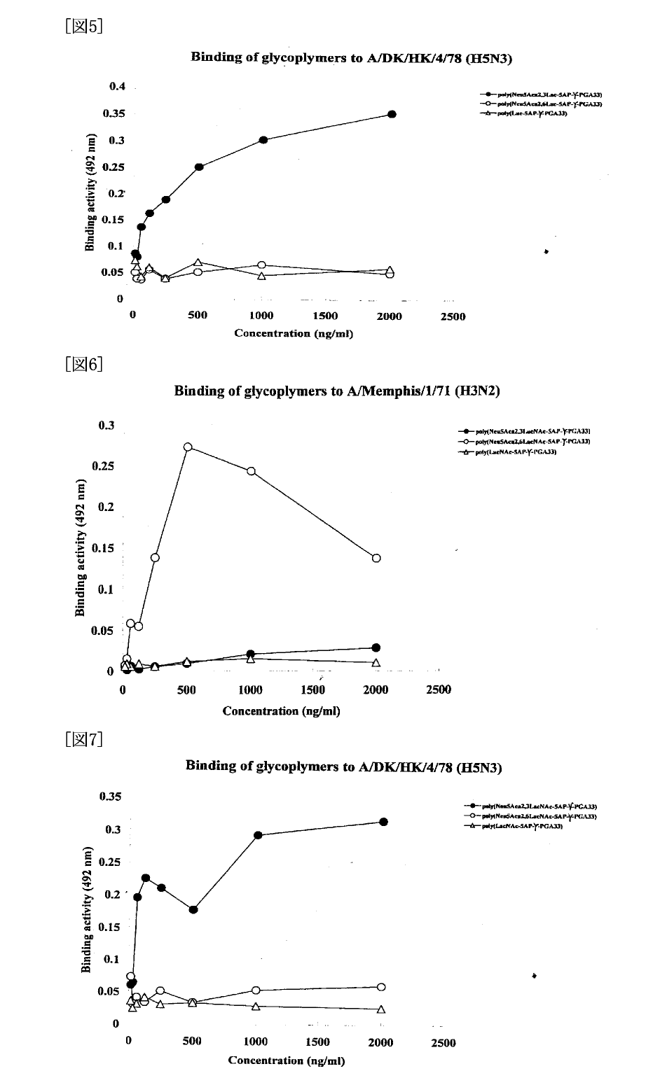

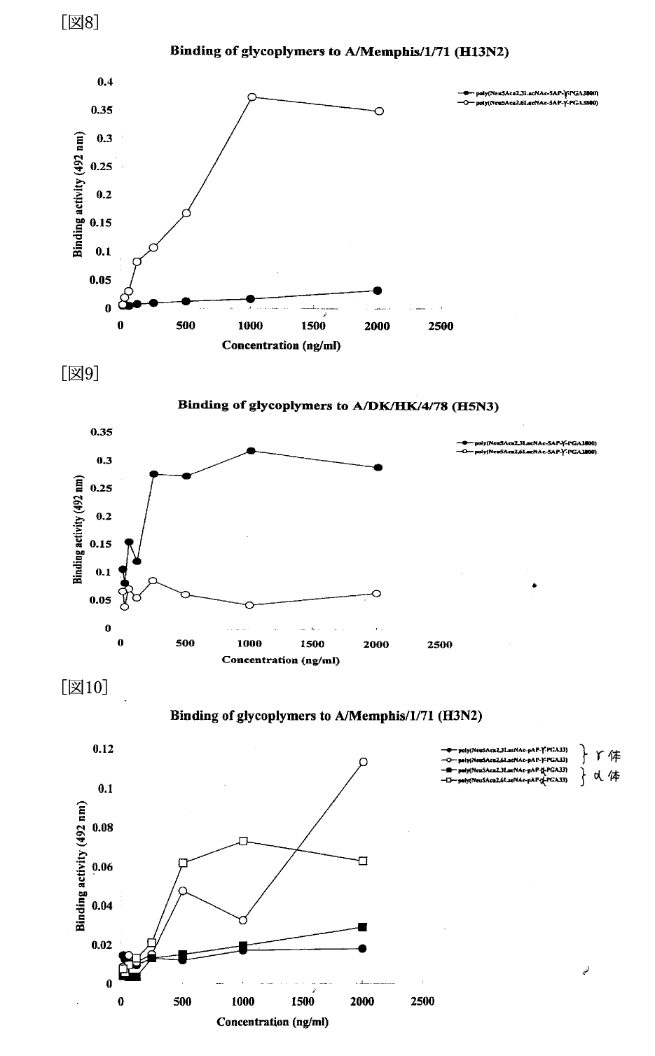

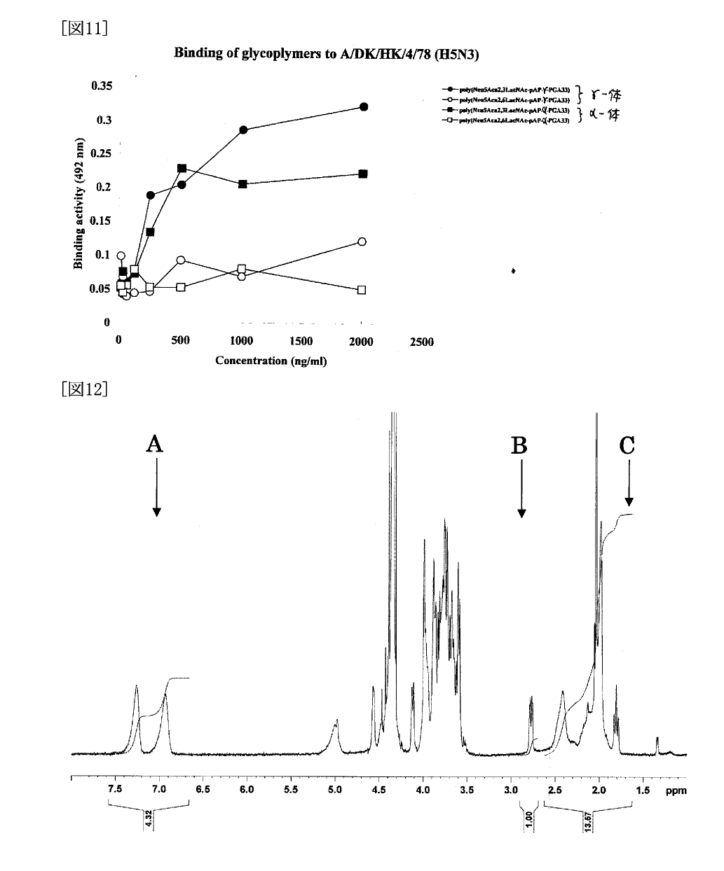

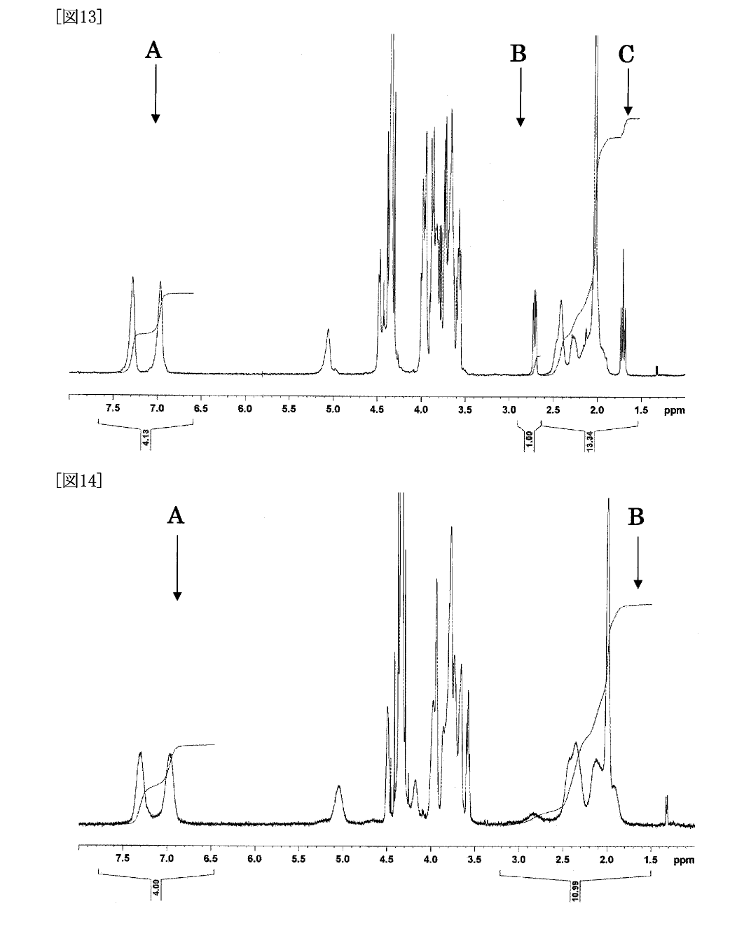

The results shown in FIG. 4 to 11 show that the avian influenza virus strongly recognized the 2-3 type of polymer with sialo-oligosaccharide, however, its recognition of the 2-6 type of polymer with sialo-oligosaccharide was weak. On the other hand, the human influenza virus strongly recognized the 2-6 type of polymer with sialo-oligosaccharide but its recognition of the 2-3 type of polymer with sialo-oligosaccharide was weak. And, by making a gradient of a binding curve for each polymer with sialo-oligosaccharide, it is possible to determine whether there has been a change in a host infected due to a virus mutation.

### Reference Example

### (1) Preparation of Para Nitro Phenylt N-acetyl-β-lactosaminide [Gal β1-4 GlcNAc β-pNP]

2.4 g of lactose and 2.3 g of para nitro phenyl N-acetyl glucopyranoside (Sigma) are dissolved in 20 mM sodium phosphate buffer (12 mL, pH 7.0) containing 20% acetonitrile, 20 units of β-galactosidase (Yamato Kasei) derived from Bacillus circulans is added and made to react for 6 hours at 40 degrees C. After this, the reaction solution is heated for 10 minutes at 95 degrees C. and after the reaction is stopped the solution is centrifugally separated and supernatants are collected. The supernatant solution is applied on a Toyopearl HW-40S column (5×100 cm), eluate is collected (20 ml/tube), and the absorbency is assayed at 300 nm using a part of the eluate and the quantity of hydrocarbons is determined. A fraction (120 mL) which contains para nitro phenyl N-acetyl-β-lactosaminide is gathered and collected and after concentration, methanol is gradually added. The separated sediment is filtered and concentrated by pressure drying so that 292 mg of para nitro phenylt N-acetyl-β-lactosaminide crystals is obtained.

### (2) Preparation of Para Amino Phenylt N-acetyl-β-lactosaminide [Gal β1-4 GlcNAc β-pAP]

100 mg of the para nitro phenylt N-acetyl-β-lactosaminide obtained in (1) is dissolved in 20 mL of methanol, 300 mg of ammonium formate and 20 mg of 10% palladium/active carbon powder is added to this solution, and made to react at 40 degrees C. At this time, the reaction is traced at regular intervals by high-performance liquid chromatography. After 40 minutes, it is confirmed that the peak of para nitro phenylt N-acetyl-p-lactosaminide has disappeared, then, the reaction solution is returned to room temperature and the reaction is stopped. The reaction solution is then filtered by sellite and filter paper and after concentrating the filtered solution is applied on a chroma trex—ODS DM1020T column chromatography process in which a column has been equibrilated with 12% methanol in advance. Fractions (30 mL/tube) are collected from the eluate and peak fractions which are expected to be an amino reduced disaccharide derivative which matched in both absorbencies of 210 nm and 300 nm are concentrated, lyophilized and 70.7 mg of para amino phenylt N-acetyl-β-lactosaminide crystals is obtained.

### (3) Preparation of Poly (Para Amino Phenylt

### N-acetyl-β-lactosaminide-L-glutamine-co-glutamine Acid) [Poly (Gal β1-4 GlcNAc β-pAP/α-PGA]

20 mg of α-Poly-L-monosodium glutamate (Sigma) is dissolved in 0.4 mL of dimethylsulfoxide, 160 mg of hexafluorophosphate benzotriazole-1-yloxytris(dimethylamino) phosphonium which has been dissolved in advance in 0.2 mL of dimethylsulfoxide and 18 mg of 1-hydroxybenzotriazole-hydrate are added and stirred at room temperature for 20 minutes. Further, 60 mg of the (para amino phenylt N-acetyl-β-lactosaminide obtained in (2) is dissolved in 0.4 mL of dimethylsulfoxide, added and stirred for 24 hours at room temperature. This reaction solution is applied on a Sephadex G-25 column (2.0×26 cm, Amersham Pharmaceutical) and eluted (speed flow 1.0 mL/min) with 0.02 M sodium phosphate buffer (pH 7.4) containing 0.1 M sodium chloride. Fractions (2.0 mL/tube) are collected from the eluate solution, and a part which is used to determine the absorbency at 485 nm using a phenol-sulfuric acid method, and fractions which contain hydrocarbon are collected (13 mL). This solution is then concentrated (2 kg/cm²) by an ultrafiltration unit equipped with a YM-3 membrane (Amicon), further lyophilized and a sample of 46 mg is obtained.

### (4) Preparation of Poly(para amino phenylt(N-acetylneuraminyl (2-3)-N-acetyl-β-lactosaminide)-L-glutamine-co-glutamine acid] [Poly (Neu5Ac α2-3Galβ 1-4 GlcNAc β-pAP/α-PGA]

10 mg of the Poly (para amino phenylt N-acetyl-β-lactosaminide-L-glutamine-co-glutamine acid) [Poly (Gal β1-4 GlcNAc β-pAP/α-PGA] obtained in (3), 15 mg of cytidine 5′-monophospho-N-acetylneuraminic acid sodium, 10 μL of 250 mM manganese chloride, 10 μL of 10% bovine serum albunin and 2 μL of alkaline phosphatase are dissolved in 950 μL of 50 mM cacodylic acid buffer (pH 6.0), 30 ml units of a2,3-(N)-sialyltransferase (rat recombinant, derived from Spodoptera frugiperda, Calbiochem) are added and a reaction is allowed to occur for 48 hours at 37 degrees C. This reaction solution is applied on a Sephadex G-25 column (2.0×26 cm, Amersham Pharmaceutical) and a final substance of 10.9 mg is obtained.

### (5) Preparation of Poly (para amino phenyl (N-acetylneuraminyl (2-6)-N-acetyl-β-lactosaminide)-L-glutamine-co-glutamine acid] [Poly (Neu5Ac α2-6Galβ 1-4 GlcNAc β-pAP/α-PGA]

5 mg of the Poly (para amino phenyl N-acetyl-β-lactosaminide-L-glutamine-co-glutamine acid) [Poly (Gal β1-4 GlcNAc β-pAP/α-PGA] obtained in (3), 7.5 mg of cytidine 5′-monophospho-N-acetylneuraminic acid sodium, 5 μL of 250 mM manganese chloride, 5 μL of 10% bovine serum albumin and 1 μL of alkaline phosphatase are dissolved in 474 μL of 50 mM cacodylic acid buffer (pH 6.0), 15 ml units of α2,6-(N)-sialyltransferase (derived from rat liver, Calbiochem) are added and a reaction is allowed to occur for 48 hours at 37 C. This reaction solution is applied on a Sephadex G-25 column (2.0×26 cm, Amersham Pharmaceutical) and a final substance of 6.3 mg is obtained.

According to the present invention, it is possible to easily determine the recognition specificity of an influenza virus for a receptor sugar chain in a simple apparatus or instrument as stated above. Therefore, according to the present invention, for example, it is possible to accurately determine the recognition specificity of an influenza virus for a receptor sugar chain even in clinical places such as examination facilities and hospitals and its application is versatile.

1-18. (canceled)

19. A method for determining the recognition specificity of a virus for a receptor sugar chain which comprises:
bringing a sample of the virus into contact with a support having a polymer with sialo-oligosaccharide immobilized on the surface thereof; and
assaying the degree of binding therein to determine the recognition specificity of the virus for the receptor sugar chain.

20. A method for determining a change in a host range caused by a virus mutation which comprises:
using a support wherein two or more different polymers with sialo-oligosaccharide are immobilized on the surface of the support(s) each of which has a different polymer with sialo-oligosaccharide immobilized on each surface;
bringing the sample of the virus into contact with each of the polymers with sialo-oligosaccharide;
assaying the degree of biding therein; and
determining a change in the host range caused by the virus mutation by comparing the results.

21. The determining method according to claim 19, wherein the sialo-oligosaccharide in the polymer with sialo-oligosaccharide is at least one sugar chain selected from a group consisting of sialyllacto-series type I sugar chain (SAα2-6(3)Galβ1-3GlcNAcβ1-), sialyllacto-series type II sugar chain (SAα2-6(3)Galβ1-4GlcNAcβ1-), sialylganglio-series sugar chain (SAα2-6(3)Galβ1-3GalNAcβ1-), and sialyl lactose sugar chain (SAα2-6(3)Gal1-4Glc-).

22. The determining method according to claim 19, wherein the polymer in the polymer with sialo-oligosaccharide is a polyglutamic acid.

23. The determining method according to claim 19, wherein the assaying the degree of binding is an immunologic assay which uses an antivirus antibody against the virus.

24. The determining method according to claim 19, wherein the virus sample is an influenza virus sample.

25. A polymer with sialo-oligosaccharide having a γ-polyglutamic acid with which a sialo-oligosaccharide is coupled, and expressed in the following formula (I): 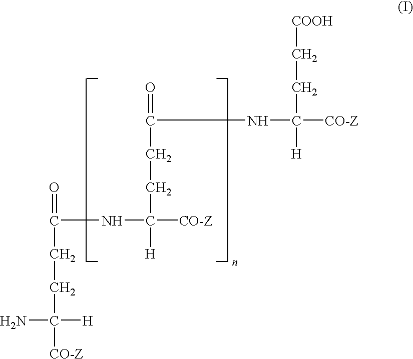
wherein n indicates an integer of 10 or more and Z is a hydroxyl group or a sialo-oligosaccharide binding site as shown in formula (II): 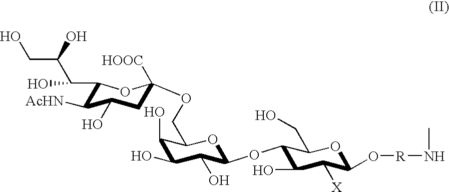
wherein Ac is an acetyl group, X is a hydroxyl group or an acetyl amino group and R is a hydrocarbon.

26. A polymer with sialo-oligosaccharide having a γ-polyglutamic acid with which a sialo-oligosaccharide is coupled, and expressed in the following formula (III): 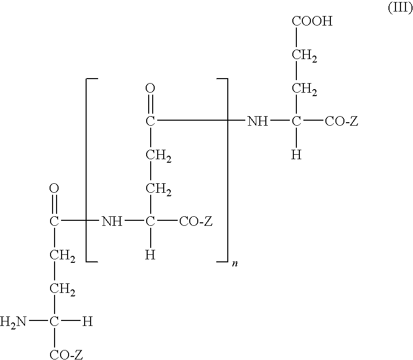
wherein n indicates an integer of 10 or more and Z is a hydroxyl group or a sialo-oligosaccharide binding site as shown in formula (IV): 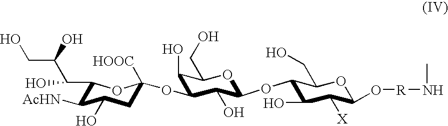
wherein Ac is an acetyl group, X is a hydroxyl group or an acetyl amino group and R is a hydrocarbon.

27. A polymer with sialo-oligosaccharide having an α-polyglutamic acid with which a sialo-oligosaccharide is coupled, and expressed in the following formula (V). 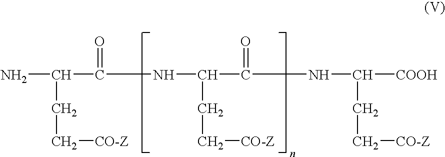
wherein n indicates an integer of 10 or more and Z is a hydroxyl group or a sialo-oligosaccharide binding site as shown in formula (VI): 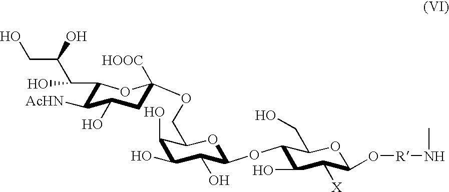
wherein Ac is an acetyl group, X is a hydroxyl group or an acetyl amino group and R′ is a hydrocarbon other than phenylene.

28. A polymer with sialo-oligosaccharide having an α-polyglutamic acid with which a sialo-oligosaccharide is coupled, and expressed in the following formula (VII): 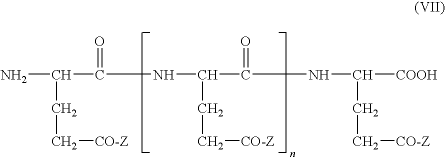
wherein n indicates an integer of 10 or more and Z is a hydroxyl group or a sialo-oligosaccharide binding site as shown in formula (VIII): 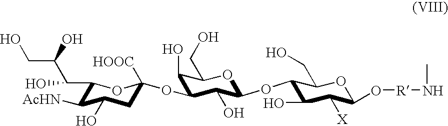
wherein Ac is an acetyl group, X is a hydroxyl group or an acetyl amino group and R′ indicates a hydrocarbon other than phenylene.

29. A manufacturing method of a polymer with sialo-oligosaccharides which comprises:
a first process wherein a desired sialo-oligosaccharide is synthesized using a glycosyltransferase;
a second process wherein the sialo-oligosaccharide synthesized in the first process is chemically coupled with a polyglutamic acid; and
a third process wherein a desired polymer with sialo-oligosaccharide is obtained by isolating and purifying the polymer with sialo-oligosaccharide synthesized in the second process.

30. The manufacturing method according to claim 29, wherein the sialo-oligosaccharide is at least one sugar chain selected from a group consisting of sialyllacto-series type I sugar chain (SAα2-6(3)Galβ1-3GlcNAcβ1-), sialyllacto-series type II sugar chain (SAα2-6(3)Galβ1-4GlcNAcβ1′-), sialylganglio-series sugar chain (SAα2-6(3)Galβ1-3GalNAcβ1-), and sialyl lactose sugar chain (SAα2-6(3)Gal1-4Glc-).

31. A support used in the determining method of claim 19 comprising a polymer with sialo-oligosaccharide immobilized on the surface of the support.

32. A support comprising a polymer with sialo-oligosaccharide immobilized on the surface thereof by ultraviolet ray irradiation, in the polymer with sialo-oligosaccharide, at least one sialo-oligosaccharide selected from a group consisting of sialyllacto-series type I sugar chain (SAα2-6(3)Galβ1-3GlcNAcβ1-), sialyllacto-series type II sugar chain (SAα2-6(3)Galβ1-4GlcNAcβ1-), sialylganglio-series sugar chain (SAα2-6(3)Galβ1-3GalNAcβ1-), and sialyl lactose sugar chain (SAα2-6(3)Gal1-4Glc-) is coupled with a polyglutamic acid.

33. The support according to claim 32, wherein the support contains a plurality of wells, and a plurality of polymers with sialo-oligosaccharide of different types being immobilized on the support.

34. A kit used in the determining method of claim 19 for determining the recognition specificity for a receptor sugar chain or a mutation of a virus comprising a support comprising a polymer with sialo-oligosaccharide immobilized on the surface thereof by ultraviolet ray irradiation, in the polymer with sialo-oligosaccharide, at least one sialo-oligosaccharide selected from a group consisting of sialyllacto-series type I sugar chain (SAα2-6(3)Galβ1-3GlcNAcβ1-), sialyllacto-series type II sugar chain (SAα2-6(3)Galβ1-4GlcNAcβ1-), sialylganglio-series sugar chain (SAα2-6(3)Galβ1-3GalNAcβ1-), and sialyl lactose sugar chain (SAα2-6(3)Gal1-4Glc-) is coupled with a polyglutamic acid.

35. The kit according to claim 34, wherein the support contains a plurality of wells, and a plurality of polymers with sialo-oligosaccharide of different types being immobilized on one support.

36. The kit according to claim 34, wherein the kit contains two or more supports, and a polymer with sialo-oligosaccharide of different type being immobilized on each of the supports.
